(19) United States
(12) Patent Application Publication
(10) Pub. No.: US 2026/0224450 A1
(43) Pub. Date: Aug. 6, 2026

(54) COSMETIC EMULSION COMPOSITION COMPRISING SPHERICAL HYDROPHOBIC SILICA AEROGEL AND PIGMENT FOR WATERY FRESH SENSATION

(71) Applicant: L'OREAL, PARIS (FR)

(72) Inventors: Arisa SATO, Kawasaki-shi (JP); Mariko OKAMOTO, Kawasaki-shi (JP); Kazunori OGAMI, Kawasaki-shi (JP)

(21) Appl. No.: 19/135,246

(22) PCT Filed: Dec. 8, 2023
(86) PCT No.: PCT/JP2023/044952

§ 371 Date: Jun. 3, 2025

(30) Foreign Application Priority Data
Dec. 15, 2022 (JP) 2022-200280
Jan. 27, 2023 (FR) 2300754

(51) Int. Cl. A61K 8/06; A61K 8/25; A61K 8/29; A61K 8/37; A61K 8/73; A61Q 1/00; A61Q 19/00

(52) U.S. Cl. CPC A61K 8/06; A61K 8/25; A61K 8/29; A61K 8/375; A61K 8/735; A61Q 1/00; A61Q 19/00; A61K 2800/5422; A61K 2800/5424; A61K 2800/61

(57) ABSTRACT

A cosmetic emulsion composition having at least one aqueous phase and at least one oily phase, contains: (a) at least one spherical hydrophobic silica aerogel; (b) at least one lipophilic thickener; (c) at least one hydrophobic surface-treated pigment; (d) at least one nonionic surfactant other than nonionic silicone surfactants; (e) at least one polyol; (f) at least one monoalcohol; and (g) at least one anionic polymer. The aqueous phase is present in an amount of 30% by weight or more relative to the total weight of the composition.

### TECHNICAL FIELD

The present invention relates to a cosmetic composition in the form of an emulsion comprising at least one spherical hydrophobic silica aerogel and at least one pigment for a watery fresh sensation.

### BACKGROUND ART

Imparting a watery and fresh texture to keratinous substances, such as skin, is one of the key features of cosmetic products, in particular skin cosmetic products. Emulsions are commonly employed as the form of liquid foundation products, since they are pleasant to use due to the feeling of freshness and the moisture that the aqueous phase can provide.

Spherical silica silylate aerogel particles have recently been developed, and it has been reported that these particles provide emulsion compositions with a long-lasting stability.

For example, JP-A-2021-102558 discloses a cosmetic composition in the form of a W/O emulsion, comprising at least one spherical hydrophobic silica aerogel and at least one ester oil selected from liquid esters of saturated or unsaturated, linear or branched C₁-C₂₆ aliphatic monoacid or polyacids and of saturated or unsaturated, linear or branched C₁-C₂₆ aliphatic monoalcohol or polyalcohol.

Also, JP-A-2021-102559 discloses a cosmetic composition comprising (i) at least one spherical hydrophobic silica aerogel and (ii) at least one composite silica particle and/or at least one hollow silica particle.

However, when compositions comprise a large amount of aqueous phase, there may be a problem that a dispersibility of pigments is decreased. There is a demand of for cosmetic compositions which can provide keratinous substances, such as skin, with a moist, watery, and fresh sensation as well as exhibit stable pigment dispersibility.

### DISCLOSURE OF INVENTION

An objective of the present invention is to provide a cosmetic composition which can provide a watery and fresh sensation with long-lasting moisture and which is stable with a good dispersibility of pigments.

The above objective can be achieved by an emulsion composition having at least one aqueous phase and at least one oily phase, comprising:
(a) at least one spherical hydrophobic silica aerogel;
(b) at least one lipophilic thickener;
(c) at least one hydrophobic surface-treated pigment;
(d) at least one nonionic surfactant selected from other than nonionic silicone surfactants;
(e) at least one polyol;
(f) at least one monoalcohol; and
(g) at least one anionic polymer,
wherein the aqueous phase is present in an amount of 30% by weight or more relative to the total weight of the composition.

The spherical hydrophobic silica aerogel may be a spherical hydrophobic aerogel of silica silylate.

The spherical hydrophobic silica aerogel may have an average circularity determined by an image analysis method of 0.8 or more, and preferably 0.82 or more, and of less than 1, preferably 0.99 or less, more preferably 0.98 or less, even more preferably 0.97 or less, still even more preferably 0.96 or less, and most preferably 0.95 or less.

The lipophilic thickener may be selected from mineral lipophilic thickeners.

The hydrophobic surface-treated pigment may be coated with isopropyl titanium triisostearate.

The polyol may comprise at least one diol and at least one polyol having three or more —OH functions, in particular triol, in combination.

The polyol may comprise at least 3% by weight of at least one diol and at least 3% by weight of at least one polyol having three or more —OH functions, in particular triol, in combination, relative to the total weight of the composition.

A number average molecular weight of the anionic polymer may range from 1,000 to 1,000,000, preferably from 5,000 to 500,000, even more preferably from 10,000 to 200,000, even more preferably from 15,000 to 100,000, and in particular from 20,000 to 50,000.

The anionic polymer may be selected from the group consisting of polysaccharides such as alginic acid, hyaluronic acid and derivatives thereof, and cellulose polymers (e.g., carboxymethylcellulose), anionic (co)polyaminoacids such as (co)polyglutamic acids, (co)poly(meth)acrylic acids, (co)polyamic acids, (co)polystyrene sulfonate, (co)poly(vinyl sulfate), dextran sulfate, chondroitin sulfate, (co)polymaleic acids, (co)polyfumaric acids, maleic acid (co)polymers.

The anionic polymer may be selected from hyaluronic acid and derivatives thereof, and a salt thereof; and preferably hyaluronic acid and acetylated hyaluronic acid, and a salt thereof.

The nonionic surfactant may be selected from esters of polyols with fatty acids.

The cosmetic composition may further comprise at least one film-forming polymer, preferably selected from silicone resin.

The cosmetic composition may further comprise at least one ad least one thickening aid selected from polyoxyalkylenated silicone and alkyl or alkylene carbonate.

The cosmetic composition according to the present invention may be a skin makeup or skin care composition, preferably a skin makeup composition, and more preferably a foundation.

The present invention also relates to a cosmetic process for a keratin substance such as skin, comprising applying the cosmetic composition according to the present invention to the keratin substance.

### BEST MODE FOR CARRYING OUT THE INVENTION

After diligent research, the inventors have discovered that a composition in the form of an emulsion comprising the components (a) to (g) according to the present invention can provide a watery and fresh sensation with long-lasting moisture and which is stable with a good dispersibility of pigments, and thus completed the invention.

Thus, the composition according to the present invention is an emulsion composition having at least one aqueous phase and at least one oily phase, comprising:
(a) at least one spherical hydrophobic silica aerogel;
(b) at least one lipophilic thickener;
(c) at least one hydrophobic surface-treated pigment;
(d) at least one nonionic surfactant other than nonionic silicone surfactants;
(e) at least one polyol;
(f) at least one monoalcohol; and
(g) at least one anionic polymer,
wherein the aqueous phase is present in an amount of 30% by weight or more relative to the total weight of the composition

Hereinafter, the composition according to the present invention will be explained in a more detailed manner.

### [Composition]

The composition of the present invention is in the form of as emulsion comprising an aqueous phase and an oily phase. The emulsion may be O/W or W/O form. In one preferred embodiment of the present invention, the composition is in the form of W/O emulsion.

The term “W/O emulsion” or “water-in-oil emulsion” means any macroscopically homogeneous composition comprising a continuous fatty or oily phase and aqueous or water phases in the form of droplets dispersed in the said fatty or oily phase. The term “O/W emulsion” or “oil-in-water emulsion” means any macroscopically homogeneous composition comprising a continuous aqueous or water phase and fatty or oily phases in the form of droplets dispersed in the aqueous or water phase.

The composition is a cosmetic composition, preferably a cosmetic composition for a keratin substance, and more preferably a skin makeup composition or a skincare composition, and in particular a skin makeup composition. The keratinous substance here means a material containing keratin as a main constituent element, and examples thereof include the skin, scalp, lips, and the like. Preferably, the composition of the present invention is used for skin, and more preferably facial skin.

In one preferred embodiment, the composition according to the present invention can be used as a liquid foundation, a make-up base, and a skin makeup cream composition, and in particular a liquid foundation. The composition can be in the form of a lotion, a milky lotion, a cream, a liquid gel, a paste, or a serum.

The composition according to the present invention can provide keratinous substances, such as skin, with moist, watery, and fresh sensation. In particular, the composition according to the present invention can provide keratinous substances with “water-splashing” sensation. The expression of “water-splashing” sensation here means a feel like a splash or a disperse of water to give a fresh feeling on the keratinous substances during an application. Such a feel can give consumers a fresh sensation.

The composition according to the present invention is stable in terms of its phase separation as well as pigments dispersion.

The composition according to the present invention comprises (a) at least one spherical hydrophobic silica aerogel; (b) at least one lipophilic thickener; (c) at least one hydrophobic surface-treated pigment; (d) at least one nonionic surfactant other than nonionic silicone surfactants; (e) at least one polyol; (f) at least one monoalcohol; and (g) at least one anionic polymer. The ingredients in the composition will be described in a detailed manner below.

### (Spherical Hydrophobic Silica Aerogel)

The composition according to the present invention comprises at least one spherical hydrophobic silica aerogel. Two or more types of spherical hydrophobic silica aerogels may be used in combination. Thus, a single type of the spherical hydrophobic silica aerogel or a combination of different types of the spherical hydrophobic silica aerogels may be used.

Aerogels are materials with high porosity. Herein, silica aerogels refer to a solid silica with a porous structure generally obtained by replacing medium included in wet silica aerogels with air by drying them while a solid network structure of the silica is maintained. The porosity represents the amount of air contained in an apparent volume of a material by a volume percentage. The spherical hydrophobic silica aerogel of the present invention may have a porosity of 60% or more, preferably 70% or more, and more preferably 80% or more.

The hydrophobic silica aerogel of the present invention is characterized in that the shape of each of the particles is spherical. Due to this spherical shape, the hydrophobic silica aerogel can provide cosmetic compositions with good smoothness. The spherical degree of the hydrophobic silica aerogel may be determined by an average circularity.

The spherical hydrophobic silica aerogel of the present invention may have an average circularity of 0.8 or more, and preferably 0.82 or more. The spherical hydrophobic silica aerogel may have an average circularity of less than 1, preferably 0.99 or less, more preferably 0.98 or less, even more preferably 0.97 or less, still even more preferably 0.96 or less, and most preferably 0.95 or less.

The “average circularity” may be determined by an image analysis method. In particular, the “average circularity” may be an arithmetic mean of circularity obtained by image analysis of a scanning electron microscope (SEM) image of no less than 2000 aerogel particles observed at a magnification of 1000 by secondary electron detection using a scanning electron microscope (SEM).

The “circularity” of each aerogel particle is a value determined by the following formula:

$$C = 4 π S / {L}^{2}$$

wherein C represents a circularity, S represents an area (projected area) of the aerogel particle in the image, and L represents a length of a periphery (perimeter) of the aerogel particle in the image. When the average circularity approaches 1, the shape of each of the particles becomes more spherical.

In the spherical hydrophobic silica aerogel of the present invention, the term “hydrophobicity” means that it is difficult for the silica aerogel particles to disperse in water. More specifically, this term means that an aerogel phase and an aqueous phase are completely separated after 1 g of the silica aerogel particles and 100 g of ion-exchange water are added to a bottle, the bottle is agitated or stirred for ten or more seconds, and the bottle is left to stand. Therefore, in one particular embodiment of the present invention, the spherical hydrophobic silica aerogel does not exhibit a water absorption property.

The spherical hydrophobic silica aerogel that may be used according to the present invention is preferably of silylated silica type (INCI name: silica silylate). Most preferably, the spherical hydrophobic silica aerogel may be those described in JP-A-2014-088307, JP-A-2014-218433, or JP-A-2018-177620.

The hydrophobicity may be obtained by reacting a hydrophobizing agent with a silanol group represented by the following formula existing on the surface of silica:

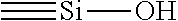

wherein the symbol “ [symbol] ” represents the remaining three valences of the Si atom, thereby converting the silanol group into a group represented by the following formula:

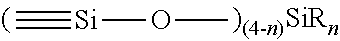

wherein n is an integer of 1 to 3; each R is independently a hydrocarbyl group; and two or more R may be the same or different from each other, where n is 2 or more.

The hydrophobizing agent may be a silylating agent. Therefore, according to one preferred embodiment, in the spherical hydrophobic silica aerogel, the silica particles may be modified at the surface by silylation. As examples of the silylating agents, mention may be made of a treating agent having one of the following formulae (1) to (3).

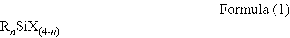

wherein n represents an integer of 1 to 3; R represents a hydrocarbyl group; X represents a group (i.e. a leaving group) which can leave a molecule by cleavage of bond with the Si atom in a reaction with a compound having a hydroxyl group; each R may be different where n is 2 or more; and each X may be different where n is 2 or less.

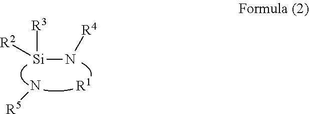

wherein R¹ represents an alkylene group; R² and R³ independently represent a hydrocarbyl group; and R⁴ and R⁵ independently represent a hydrogen atom or a hydrocarbyl group.

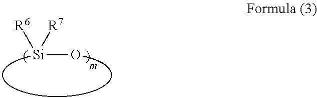

wherein R⁶ and R⁷ independently represent a hydrocarbyl group; m represents an integer of 3 to 6; each R⁶ may be different when there are two or more R⁶; and each R⁷ may be different when there are two or more R⁷.

In the above formula (1), R is a hydrocarbyl group, preferably a hydrocarbyl group having a carbon number of 1 to 10, more preferably a hydrocarbyl group having a carbon number of 1 to 4, and especially preferably a methyl group.

As examples of the leaving group represented by X, mention may be made of halogen atoms such as chlorine and bromine; alkoxy groups such as methoxy group and ethoxy group; groups represented by —NH—SiR₃ (wherein the definition of R is the same as that of R in the formula (1)).

Specific examples of the hydrophobizing agent represented by the above formula (1) include: chlorotrimethylsilane, dichlorodimethylsilane, trichloromethylsilane, monomethyltrimethoxysilane, monomethyltriethoxysilane, and hexamethyldisilazane.

Most preferably, chlorotrimethylsilane, dichlorodimethylsilane, trichloromethylsilane, and/or hexamethyldisilazane may be used from the viewpoint of favorable reactivity.

The number of bonds of the Si atom with the silanol group on the silica framework varies depending on the number (4-n) of the leaving group X. For example, if n is 2, the following bonding will occur:

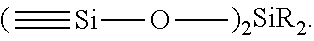

If n is 3, the following bonding will occur:

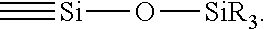

In this manner, the silanol groups may be silylated, and thereby hydrophobization may be carried out.

In the above formula (2), R¹ may be an alkylene group, preferably an alkylene group having a carbon number of 2 to 8, and especially preferably an alkylene group having a carbon number of 2 to 3.

In the above formula (2), R² and R³ are independently a hydrocarbyl group, and the same preferable groups as those of R in the formula (1) can be raised. R⁴ represents a hydrogen atom or a hydrocarbyl group, and when it is a hydrocarbyl group, the same preferable groups as those of R in the formula (1) can be raised. When a gel of silica is treated with the compound (cyclic silazane) represented by the formula (2), cleavage of Si—N bonds will occur by the reaction with silanol groups, and therefore the following bonding will occur on the surface of the silica framework in the gel:

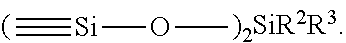

In this way, the silanol group may be silylated by the cyclic silazanes of the above formula (2) as well, and thereby hydrophobization may be carried out.

Specific examples of the cyclic silazanes represented by the above formula (3) include hexamethylcyclotrisilazane, and octamethylcyclotetrasilazane.

In the above formula (3), R⁶ and R⁷ are independently a hydrocarbyl group, and the same preferable groups as those of R in the formula (2) can be raised. m represents an integer of 3 to 6. When a gel of silica is treated with the compound (cyclic siloxane) represented by the formula (3), the following bonding will occur on the surface of the silica framework in the gel:

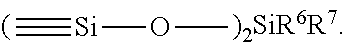

In this way, silanol groups may be silylated by the cyclic siloxanes of the above formula (3) as well, and thereby hydrophobization may be carried out.

Specific examples of the cyclic siloxanes represented by the above formula (3) include hexamethylcyclotrisiloxane, octamethylcyclotetrasiloxane, and decamethylcyclopentasiloxane.

The spherical hydrophobic silica aerogel may be prepared by producing a sol of silica, turning the sol into a gel, aging the gel, washing the aged gel, replacing water in the washed gel with a solvent, treating the gel with a hydrophobizing agent, and dying the hydrophobized silica.

The spherical hydrophobic silica aerogel may have a specific surface area determined by BET method of 200 m²/g or more, preferably 400 m²/g or more, and more preferably 500 m²/g or more, and may have a specific surface area determined by BET method of 1,200 m²/g or less, preferably 1,000 m²/g or less, and more preferably 800 m²/g or less.

In the present invention, the “specific surface area determined by BET method” means a value determined by: drying a sample for measurement at 200° C. for no less than three hours under a reduced pressure of no more than 1 kPa; thereafter measuring an adsorption isotherm of only a nitrogen adsorption side at liquid nitrogen temperature; and analyzing the adsorption isotherm by the BET method. The pressure range used for the analysis is a relative pressure of 0.1 to 0.25.

The spherical hydrophobic silica aerogel may have a pore volume determined by BJH method of 1 ml/g or more, preferably 2 ml/g or more, and more preferably 3 ml/g or more, and may have a pore volume determined by BJH method of 10 ml/g or less, preferably 8 ml/g or less, and more preferably 7 ml/g or less. The spherical hydrophobic silica aerogel may have a peak pore radius determined by BJH method of 5 nm or more, preferably 10 nm or more, and more preferably 12 nm or more, and may have a peak pore radius determined by BJH method of 50 nm or less, preferably 40 nm or less, and more preferably 30 nm or less.

The “pore volume determined by BJH method” refers to a pore volume which is derived from a pore having a pore radius of 1 nm to 100 nm obtained by analyzing, by the BJH method (Barrett, E. P.; Joyner, L. G.; Halenda, P. P., J. Am. Chem. Soc. 73, 373 (1951)), the adsorption isotherm of the nitrogen adsorption side obtained in the same manner as explained in the above “specific surface area determined by BET method”. The “peak pore radius determined by BJT method” refers to a value of a pore radius which gives a peak in a pore distribution curve (volume distribution curve) which is plotted taking on the vertical axis differentiation of the cumulative pore volume by the logarithm of the pore radius obtained by analyzing, by the BJH method, the adsorption isotherm of the nitrogen adsorption side obtained in the same manner as above, and taking the pore radius on the horizontal axis.

The spherical hydrophobic silica aerogel may have an average particle size of 0.5 μm or more, preferably 1 μm or more, and more preferably 2 μm or more, and may have an average particle size by image analysis method of 30 μm or less, preferably 20 μm or less, and more preferably 15 μm or less.

The “average particle size” here can be measured by an image analysis method. Specifically, the value of “average particle size” is an arithmetic mean of equivalent circle diameters which can be obtained by image analysis of a scanning electron microscope (SEM) image of, for example, no less than 2000 aerogel particles observed at a magnification of 1000 by secondary electron detection using a scanning electron microscope (SEM). The “equivalent circle diameter” of each aerogel particle is a diameter of a circle having an area equal to the area (projected area) of the aerogel particle in the image.

Preferably, the spherical hydrophobic silica aerogel may have an oil-absorbing capacity, which can be measured at the wet point, of 2 ml/g or more, preferably 3 ml/g or more, more preferably 4 ml/g or more, and most preferably from 5 ml/g or more, and may have an oil-absorbing capacity, measured at the wet point, of 12 ml/g or less, preferably 11 ml/g or less, more preferably 10 ml/g or less, and most preferably 8 ml/g or less.

The oil-absorbing capacity measured at the wet point, noted Wp, corresponds to the amount of oil that needs to be added to 100 g of particles in order to obtain a homogeneous paste. It can be measured according to the wet point method or the method for determining the oil uptake of a powder described in standard NF T 30-022. The oil uptake can correspond to the amount of oil adsorbed onto the available surface of the powder and/or absorbed by the powder by measuring the wet point, described below.

An amount of m=2 g of powder is placed on a glass plate, and an oil (such as ester oil, oleic acid, or silicone oil) is then added drop-wise. After addition of 4 to 5 drops of oil to the powder, mixing is performed using a spatula, and addition of oil is continued until a conglomerate of oil and powder has formed. At this point, the oil is added one drop at a time and the mixture is then triturated with the spatula. The addition of oil is stopped when a firm, smooth paste is obtained. This paste must be able to be spread on the glass plate without cracking or forming lumps. The volume Vs (expressed in ml) of oil used is then noted. The oil uptake corresponds to the ratio Vs/m.

Otherwise, an oil-absorbing capacity can be measured in accordance with JIS-K6217-4.

In one preferred embodiments of the present invention, the (a) spherical hydrophobic silica aerogels are those described in JP-A-2014-088307, JP-A-2014-218433, or JP-A-2018-177620.

The spherical hydrophobic silica aerogel(s) may be present in an amount of 0.05% by weight or more, preferably 0.1% by weight or more, more preferably 0.2% by weight or more, and even more probably 0.3% by weight or more, and/or may be present in an amount of 3% by weight or less, preferably 2% by weight or less, more preferably 1.5% by weight or less, and most preferably 1% by weight or less, relative to the total weight of the composition.

The amount of the spherical hydrophobic silica aerogel(s) in the composition according to the present invention may be from 0.05% to 3% by weight, preferably from 0.1% to 2% by weight, more preferably from 0.2% to 1.5% by weight, and even more preferably 0.3% to 1% by weight, relative to the total weight of the composition.

### Oily Phase

The oily phase of the present invention comprises at least the (b) lipophilic gelling agent and the (c) hydrophobic surface-treated pigment. The oil phase may include any lipophilic, liposoluble or lipodispersible ingredients in addition to the ingredients (b) and (c).

The amount of the oily phase is not particularly limited. In general, the amount of the oily phase may be 5% by weight or more, preferably 10% by weight or more, more preferably 20% by weight or more, and/or may be 60% by weight or less, preferably 55% by weight or less, and more preferably 50% by weight or less relative to the total weight of the composition.

The amount of the oily phase in the composition according to the present invention may be from 5% to 60% by weight, preferably from 10% to 55% by weight, and more preferably from 20% to 50% by weigh, relative to the total weight of the composition.

### (Lipophilic Thickener)

The composition according to the present invention comprises at least one lipophilic thickener. Two or more lipophilic thickeners may be used in combination. Thus, a single type of the lipophilic thickener or a combination of different types of the lipophilic thickeners may be used.

For the purposes of the present invention, the term “lipophilic” here can mean substances having a solubility of at least 1 g/L, preferably at least 10 g/L, and more preferably at least 100 g/L, in an oil at room temperature (25° C.) and atmospheric pressure (105 Pa). In another aspect, the term “lipophilic” can refer to substances which are not soluble in water or which have a solubility of 1 g/L or less, or 0.1 g/L or less in water, at 25° C. and atmospheric pressure.

The term “lipophilic thickener” means an agent, inorganic or organic, in a particulate form or not, able to gel the oils of the composition. The term “particulate lipophilic thickener” means a lipophilic thickener in the form of particles or of crystals (particulate or crystalline).

The lipophilic thickener(s) used in the composition according to the invention may be chosen from mineral lipophilic thickeners and organic lipophilic thickeners, and mixtures of these compounds. The lipophilic thickener is preferably particulate.

The mineral lipophilic thickeners that may be used in the composition according to the invention are preferably mineral particles constituted essentially of mineral oxides and/or hydroxides.

These particles are preferably insoluble in water at room temperature (25° C.). The term “insoluble” means a solubility of less than 0.5% by weight.

Preferably, the number-average primary size of these mineral particles ranges from 0.01 to 500 μm, it preferably ranges from 0.1 to 200 μm, and even more preferentially it ranges from 1 to 100 μm.

For the purposes of the present invention, the term “primary particle size” means the maximum dimension that it is possible to measure between two diametrically opposite points on an individual particle.

The size of the mineral particles may be determined by transmission electron microscopy or by measuring the specific surface area via the BET method or by laser particle size analysis.

The mineral particles that may be used in accordance with the invention may be in various forms, for example in the form of spheres, needles, flakes or platelets.

In a preferred variant of the invention, the mineral lipophilic thickener(s) are platelet-shaped particles.

The mineral lipophilic thickener(s) that may be used in the cosmetic composition according to the invention may preferably be chosen from silicas and silicates.

The silicates of the invention may be natural or chemically modified (or synthetic).

Silicates correspond to optionally hydrated silica in which some of the silicon atoms are replaced with metal cations such as Al³⁺, B³⁺, Fe³⁺, Ga³⁺, Be²⁺, Zn²⁺, Mg²⁺, Co³⁺, Ni³⁺, Na⁺, Li⁺, Ca²⁺, Cu²⁺.

More particularly, the silicates that may be used in the context of the invention are chosen from clays of the smectite family such as montmorillonites, hectorites, bentonites, beidellites and saponites, and also of the vermiculite, stevensite and chlorite families.

These clays may be of natural or synthetic origin. Clays that are cosmetically compatible and acceptable with keratin materials are preferably used.

The silicate may be chosen from montmorillonite, bentonite, hectorite, attapulgite and sepiolite, and mixtures thereof. The silicate(s) are preferably chosen from bentonites and hectorites.

The silicates may be modified with a compound chosen from quaternary amines, tertiary amines, amine acetates, imidazolines, amine soaps, fatty sulfates, alkylarylsulfonates and amine oxides, and mixtures thereof.

As silicates that are suitable for use, mention may be made of quaternium-18 bentonites, such as those sold under the names Bentone 3, Bentone 38 and Bentone 38V by Rheox, Tixogel VP by United Catalyst and Claytone 34, Claytone 40 and Claytone XL by Southern Clay; stearalkonium bentonites, such as those sold under the names Bentone 27 by Rheox, Tixogel LG by United Catalyst and Claytone AF and Claytone APA by Southern Clay; quaternium-18/benzalkonium bentonites, such as those sold under the names Claytone HT and Claytone PS by Southern Clay; quaternium-18 hectorites, such as those sold under the names Bentone Gel DOA, Bentone Gel ECO5, Bentone Gel EUG, Bentone Gel IPP, Bentone Gel ISD, Bentone Gel SS71, Bentone Gel VS8 and Bentone Gel VS38 by Rheox, and Simagel M and Simagel SI 345 by Biophil.

The silicates that may be used in the composition according to the invention may be chosen, in particular, from modified hectorites such as hectorite modified with a C₁₀-C₁₂ fatty acid ammonium chloride, especially distearyldimethylammonium chloride and stearylbenzyldimethylammonium chloride.

As explained previously, the mineral lipophilic thickener(s) that may be used in the composition according to the invention may be silicas.

The silicas that may be used in the composition according to the invention are fumed silicas.

The fumed silicas may be obtained by high-temperature hydrolysis of a volatile silicon compound in an oxyhydrogen flame, producing a finely divided silica. This process makes it possible in particular to obtain hydrophilic silicas bearing a large number of silanol groups at their surface. Such hydrophilic silicas are sold, for example, under the names Aerosil 130®, Aerosil 200®, Aerosil 255®, Aerosil 300® and Aerosil 380® by the company Degussa, and Cab-O-Sil HS-5®, Cab-O-Sil EH-5®, Cab-O-Sil LM-130®, Cab-O-Sil MS-55® and Cab-O-Sil M-5® by the company Cabot.

It is possible to chemically modify the surface of said silicas, via a chemical reaction generating a reduction in the number of silanol groups. It is especially possible to substitute silanol groups with hydrophobic groups: a hydrophobic silica is then obtained.

The hydrophobic groups may be:
(a) trimethylsiloxy groups, which are obtained especially by treating fumed silica in the presence of hexamethyldisilazane. Silicas thus treated are known as Silica silylate according to the CTFA (6th Edition, 1995). They are sold, for example, under the references Aerosil R812® by the company Degussa, and Cab-O-Sil TS-530® by the company Cabot;
(b) dimethylsilyloxy or polydimethylsiloxane groups, which are obtained in particular by treating fumed silica in the presence of polydimethylsiloxane or dimethyldichlorosilane.

Silicas thus treated are known as silica dimethyl silylate according to the CTFA (6th Edition, 1995). They are sold, for example, under the references Aerosil R972® and Aerosil R974® by the company Degussa, and Cab-O-Sil TS-610® and Cab-O-Sil TS-720® by the company Cabot.

Preferentially, the mineral lipophilic thickeners are chosen from hectorites modified with a C₁₀-C₁₂ fatty acid ammonium chloride, especially distearyldimethylammonium chloride and stearylbenzyldimethylammonium chloride, and hydrophilic fumed silicas such as the hydrophilic silicas sold under the name Aerosil 200®.

More preferentially, the mineral lipophilic thickeners are chosen from hectorites modified with a C₁₀-C₁₂ fatty acid ammonium chloride, especially hectorite modified with distearyldimethylammonium chloride (or disteardimonium hectorite), such as the product sold under the name Bentone 38VCG by Elementis, and the hectorite modified with stearylbenzyldimethylammonium chloride, such as the product sold under the name Bentone 27V by Elementis.

As explained previously, the lipophilic thickener(s) that may be used in the composition according to the invention may also be chosen from organic lipophilic thickeners.

Preferably, the organic lipophilic thickener(s) are chosen from semi-crystalline polymers, non-silicone polyamides, silicone polyamides, saccharide or polysaccharide monoalkyl or polyalkyl esters, N-acylamino acid amide derivatives, polymers comprising an alkylene and/or styrene block, such as polystearyl acrylates, elastomeric organopolysiloxanes, solid fatty esters, in particular C₈-C₃₀ and preferably C₁₈-C₂₄ fatty acid esters, and mixtures of these compounds. These copolymers may be diblock, triblock or multi-block polymers, radial-block polymers, also known as star copolymers, or alternatively comb polymers.

Among the C₈-C₃₀ and preferably C₁₈-C₂₄ fatty acid esters, mention may be made of mono-, di- or triesters of C₈-C₃₀ and preferably C₁₈-C₂₄ fatty acids and of polyols, more particularly mono-, di- or triesters of C₈-C₃₀ and preferably C₁₈-C₂₄ fatty acids and of glycerol. Use may especially be made of a mixture of these compounds such as a mixture of mono-, di- and triesters of behenic acid and of glycerol.

Most particularly, the organic lipophilic thickener(s) are chosen from semi-crystalline polymers, non-silicone polyamides, silicone polyamides, polymers comprising an alkylene and/or styrene block, such as polystearyl acrylates, solid fatty esters, in particular C₈-C₃₀ and preferably C₁₈-C₂₄ fatty acid esters, and mixtures of these compounds.

Even more preferentially, the organic lipophilic thickener(s) are chosen from C₈-C₃₀ and preferably C₁₈-C₂₄ fatty acid esters and mixtures thereof, better still esters of C₈-C₃₀ and preferably C₁₈-C₂₄ fatty acids and of polyols, more particularly mono-, di- or triesters of C₈-C₃₀ and preferably C₁₈-C₂₄ fatty acids and of glycerol.

The lipophilic thickener(s) may be present in an amount of 0.1% by weight or more, preferably 0.2% by weight or more, more preferably 0.5% by weight or more, and/or may be present in an amount of 5% by weight or less, preferably 3% by weight or less, and more preferably 2% by weight or less, relative to the total weight of the composition.

The amount of the lipophilic thickener(s) in the composition according to the present invention may be from 0.1% to 5% by weight, preferably from 0.2% to 3% by weight, and more preferably from 0.5% to 2% by weight, relative to the total weight of the composition.

### (Hydrophobic Surface-Treated Pigment)

The composition according to the present invention comprises at least one hydrophobic surface-treated pigment. Two or more hydrophobic surface-treated pigments may be used in combination. Thus, a single type of hydrophobic surface-treated pigment or a combination of different types of hydrophobic surface-treated pigments may be used.

The term “pigments” means white or coloured, mineral or organic particles, which are insoluble in an aqueous medium, and which are intended to colour and/or opacify the resulting composition. These pigments may be white or coloured, and mineral and/or organic.

According to a particular embodiment, the pigments used in the present invention are chosen from mineral pigments. The term “mineral pigment” means any inorganic pigment.

Among the mineral pigments that are useful in the present invention, mention may be made of metal oxides, such as zirconium oxide or cerium oxide, titanium dioxide and also zinc oxide, iron oxide (black, yellow or red) or chromium oxide, as well as manganese violet, ultramarine blue, chromium hydrate and ferric blue, and metal powders, for instance aluminum powder or copper powder, or any combinations thereof. The following mineral pigments may also be used: Ta₂O₅, Ti₃O₅, Ti₂O₃, TiO, ZrO₂ as a mixture with TiO₂, ZrO₂, Nb₂O₅, CeO₂, or ZnS. In the context of the present invention, the mineral pigments are more particularly iron oxide and/or titanium dioxide.

The mean particle size of the coated pigment is in general 100 nm or more. The mean particle size of the coated pigment according to the present invention may range from 100 nm to 25 μm, preferably from 200 nm to 10 μm. For the purpose of the present invention, the D50 size, or volume average size, corresponds to the particle size defined such that 50% by volume of the particles have a size greater than D50. The volume average size may be assessed by light diffraction using a Malvern MasterSizer laser particle size analyzer, said particles to be evaluated being dispersed in a liquid medium, for instance octyldodecyl neopentanoate.

The pigments may also be nacres and/or particles with metallic glints. The term “nacres” should be understood as meaning iridescent or non-iridescent coloured particles of any shape, especially produced by certain molluscs in their shell or alternatively synthesized, which have a colour effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles, at the surface of which are superposed at least two successive layers of metal oxides and/or of organic dyestuffs.

The pigment of the present invention is hydrophobic surface-treated. In other words, the pigment of the present invention has a hydrophobic coating. The hydrophobic coating preferably comprises at least one hydrophobic compound, preferably chosen from fatty substances, silicone surface agents, fluorinated surface agents, fluorosilicone surface agents, metallic soaps, N-acylated amino acids and salts thereof, lecithin and derivatives thereof, isopropyl titanium triisostearate, isostearyl sebacate, phospholipids and mixtures thereof.

In one preferred embodiment of the present invention, the hydrophobic surface-treated pigment is coated with isopropyl titanium triisostearate.

The type of the fatty substance is not limited. A single type of fatty substance or a combination of different types of fatty substances may be used.

As used herein, the term “fatty substance” means an organic compound that is insoluble in water at room temperature and atmospheric pressure (760 mmHg) (solubility of less than 5%, preferably 1% and even more preferably 0.1%). The fatty substance may include, in its structure, at least one sequence of at least two siloxane groups or at least one hydrocarbon-based chain containing at least 6 carbon atoms. The fatty substance is generally a lipophilic substance, and may be soluble in organic solvents, for instance, chloroform, ethanol, benzene, liquid petroleum jelly, or decamethylcyclopentasiloxane, at room temperature and atmospheric pressure.

In one embodiment, the fatty substance is an oil. As used herein, “oil” means any fatty substance that is not in solid form at room temperature and atmospheric pressure. Any oils that are suitable for cosmetic use may be used in the present invention. Suitable oils may include both volatile and non-volatile oils.

The fatty substance may be selected from hydrocarbon oils, silicone oils, fluoro oils and wax, preferably from hydrocarbon oils and wax.

The hydrocarbon oils may be hydrocarbon-based oils of animal, plant, mineral, or synthetic origin. As used herein, “hydrocarbon-based oil” means any oil, linear or branched, volatile or nonvolatile, predominantly containing carbon and hydrogen atoms, and possibly ester, ether or fluoro groups. It is preferable that the hydrocarbon oils be selected from hydrocarbon-based oils of plant or mineral origin.

Examples of hydrocarbon-based oils of animal origin may include, but are not limited to, squalene, perhydrosqualene and squalane.

The hydrocarbon-based oils of plant origin may include oils known as plant oils or vegetable oils. Examples of hydrocarbon-based oils of plant origin may include, but are not limited to, jojoba oil, avocado oil, olive oil, Rosa canina oil, sweet almond oil, coriander oil, groundnut oil, coconut oil, shea butter oil, palm oil, linseed oil, camellia oil, macadamia nut oil, sunflower oil, apricot oil, soybean oil, arara oil, hazelnut oil, corn oil, mink oil, sasanqua oil, castor oil, safflower oil, almond oil, grapeseed oil, sesame oil, soybean oil, peanut oil, sesame seed oil, canola oil, apricot oil, mango oil, walnut oil, poppy seed oil, barley oil, rye oil, and mixtures thereof. Among them, jojoba oil, particularly jojoba seed oil, avocado oil, olive oil, particularly olive fruit oil, Rosa canina oil, particularly Rosa canina fruit oil and mixtures.

The hydrocarbon-based oils of mineral or synthetic origin may be volatile or non-volatile, and examples of such oils may include, but are not limited to, liquid paraffins, petroleum jelly, liquid petroleum jelly (mineral oil), hydrogenated isoparaffin or hydrogenated polyisobutenes such as Parleam®, perhydrosqualene, polydecenes, isohexadecane, isododecane, and mixtures thereof. Among them, mineral oil may be particularly preferably used.

In one embodiment, the fatty substance is a wax. As used herein, “wax” means a fatty substance which is substantially solid at room temperature under atmospheric pressure, and has a melting point of, for example, 30° C. or more. Representative examples of such waxes may include: waxes of animal, plant, or mineral origin, for instance candelilla wax, beeswax, carnauba wax, ouricury wax, sugar cane wax, paraffin wax, lanolin wax, montan wax, and ozokerites; hydrogenated oils such as hydrogenated jojoba oil; waxes of synthetic origin, such as polyethylene waxes; and silicone waxes such as alkyl- and alkoxy-poly(di)methylsiloxanes or poly(di)methyl-siloxane esters. Among them, candelilla wax may be preferably used.

The fatty substance may also be selected from fatty acids, synthetic esters and ethers, fatty alcohols and fatty amides.

As examples of fatty acids, mention may be made of, C₁₂-C₂₂ higher fatty acids, e.g., C₁₂-C₂₂ saturated fatty acids such as stearic acid; C₁₂-C₂₂ unsaturated fatty acids such as oleic acid, linoleic acid, and linolenic acid; and mixtures thereof.

Examples of synthetic esters and ethers may include, but are not limited to, oils of formulae R₁COOR₂ and R₁—OR₂ in which R₁ represents the residue of a fatty acid or of a fatty alcohol including from 8 to 29 carbon atoms, and R₂ represents a branched or unbranched hydrocarbon chain comprising from 3 to 30 carbon atoms. For example, purcellin oil, 2-ethylhexyl palmitate (or octyl palmitate), isopropyl lanolate, isopropyl laurate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerylthrityl tetraisostearate; or lipophilic derivatives of amino acids, such as isopropyl lauroyl sarcosinate, may be used.

Other exemplary esters may include, for example, 2-ethylhexyl caprate/caprylate (or octyl caprate/caprylate), ethyl laurate, butyl laurate, hexyl laurate, isohexyl laurate, methyl myristate, ethyl myristate, butyl myristate, isobutyl myristate, isopropyl myristate, 2-octyldodecyl myristate, 2-ethylhexyl monococoate (or octyl monococoate), methyl palmitate, ethyl palmitate, isopropyl palmitate, isobutyl palmitate, butyl stearate, isopropyl stearate, isobutyl stearate, 2-ethylhexyl stearate (or octyl stearate), isopropyl isostearate, isocetyl stearate, isostearyl isostearate, 2-ethylhexyl pelargonate (or octyl pelargonate), 2-ethylhexyl hydroxystearate (or octyl hydroxystearate), decyl oleate, diisopropyl adipate, bis(2-ethylhexyl) adipate (or dioctyl adipate), diisocetyl adipate, 2-ethylhexyl succinate (or octyl succinate), diisopropyl sebacate, 2-ethylhexyl malate (or octyl malate), pentaerythrityl caprate/caprylate, 2-ethylhexyl hexanoate (or octyl hexanoate), octyldodecyl octanoate, isodecyl neopentanoate, isostearyl neopentanoate, octyldodecyl neopentanoate, isononyl isononanoate, isotridecyl isononanoate, cetearyl isononanoate, isodecyl isononanoate, isotridecyl isononanoate, lauryl lactate, myristyl lactate, cetyl lactate, myristyl propionate, 2-ethylhexyl 2-ethylhexanoate (or octyl 2-ethylhexanoate), 2-ethylhexyl octanoate (or octyl octanoate), and cetyl 2-ethylhexanoate.

The fatty alcohol that may be useful in the present invention may be non-alkoxylated, saturated or unsaturated, linear or branched, and have from 6 to 30 carbon atoms and more particularly from 8 to 30 carbon atoms. The fatty alcohols which may be preferably used in the present invention are in a liquid form, or not solid, at room temperature. Examples of the fatty alcohol may include, but are not limited to, cetyl alcohol, stearyl alcohol and their mixture (cetylstearyl alcohol), isostearyl alcohol, octyldodecanol, 2-butyloctanol, 2-hexyldecanol, 2-undecylpentadecanol, oleic alcohol and linoleic alcohol. Myristyl alcohol, lauryl alcohol, tridecyl alcohol, pentadecyl alcohol, arachidyl alcohol, behenyl alcohol and myricyl alcohol may also be used.

Examples of fatty amides include isopropyl lauroyl sarcosinate.

The fatty material may also be volatile or non-volatile silicone oils. Examples of volatile or non-volatile silicone oils that are preferably used in the present invention may include, but are not limited to, cyclopolydimethylsiloxanes (cyclomethicones) such as cyclohexasiloxane, polydimethylsiloxanes including alkyl, alkoxy or phenyl groups, phenyl silicones such as phenyl trimethicones, phenyl dimethicones, phenyltrimethylsiloxydiphenylsiloxanes, diphenyl dimethicones, diphenylmethyldiphenyltrisiloxanes, 2-phenylethyltrimethyl siloxysilicates and polymethylphenylsiloxanes.

The fluoro oils that may be suitable for the present invention may include perfluoromethylcyclopentane and nonafluoromethoxybutane.

Preferably, the fatty substance is selected from the group consisting of hydrocarbon oils, waxes and mixtures thereof, preferably be selected from the group consisting of hydrocarbon-based oils of plant or mineral origin, waxes and mixtures thereof, more preferably selected from the group consisting of jojoba oil, particularly jojoba seed oil, avocado oil, olive oil, particularly olive fruit oil, rosa canina oil, particularly rosa canina fruit oil, candelilla wax and mixtures thereof.

The silicone surface agents may be chosen from organopolysiloxanes, silane derivatives, silicon-acrylate copolymers, silicone resins, and mixtures thereof. The term organopolysiloxane denotes a compound having a structure comprising an alternation of silicon atoms and oxygen atoms and comprising organic radicals bound with silicon atoms.

The fluorinated surface agents may be chosen from perfluoroalkyl phosphates, perfluoropolyethers, polytetrafluoropolyethylene (PTFE), perfluoroalkanes, perfluoroalkyl silazanes, hexafluoropropylene polyoxides or polyorganosiloxanes comprising perfluoroalkyl perfluoropolyether groups. The term perfluoroalkyl radical denotes an alkyl radical wherein all the hydrogen atoms have been replaced by fluorine atoms.

The fluorosilicone surface agents may be chosen from perfluoroalkyl dimethicones, perfluoroalkyl silanes and perfluoroalkyl trialkoxysilanes.

The hydrophobic compound may also be chosen from metallic soaps, such as aluminum dimyristate, the aluminum salt of hydrogenated tallow glutamate. As metallic soaps, mention may particularly be made of metallic soaps of fatty acids having 12 to 22 carbon atoms, and particularly those having 12 to 18 carbon atoms. The metal of the metallic soap may particularly be zinc or magnesium. As a metallic soap, zinc laurate, magnesium stearate, magnesium myristate, zinc stearate, and mixtures thereof may be used.

The hydrophobic compound may also be chosen from N-acylated amino acids or salts thereof which may comprise an acyl group having 8 to 22 carbon atoms, such as for example a 2-ethylhexanoyl, caproyl, lauroyl, myristoyl, palmitoyl, stearoyl or cocoyl group. The amino acid may be for example lysine, glutamic acid or alanine. The salts of these compounds may be aluminum, magnesium, calcium, zirconium, zinc, sodium or potassium salts. In this way, according to one particularly preferred embodiment, an N-acylated amino acid derivative may be particularly a glutamic acid derivative and/or any of the salts thereof, and more particularly a stearoyl glutamate, such as, for example, aluminum stearoyl glutamate.

The amount of the hydrophobic compound, preferably fatty substance(s) in the hydrophobic coating is 50% by weight or more, preferably 80% by weight or more, and more preferably 95% by weight or more, relative to the total weight of the hydrophobic coating.

In one preferred embodiment of the present invention, the hydrophobic surface-treated pigment are selected from pigments coated with isopropyl titanium triisostearate, i.e., the surface of the pigments is coated or treated with isopropyl titanium triisostearate.

The hydrophobic surface-treated pigments may be prepared according to surface treatment techniques of chemical, electronic, mechanochemical or mechanical nature that are well known to those skilled in the art. Commercial products may also be used. The surface agent of isopropyl titanium triisostearate may be absorbed, adsorbed or grafted onto the surface of the pigments by evaporation of solvent, chemical reaction and creation of a covalent bond.

The hydrophobic surface-treated pigment(s) may be present in an amount of 1% by weight or more, preferably 5% by weight or more, and more preferably 10% by weight or more, and/or may be present in an amount of 30% by weight or less, preferably 25% by weight or less, and more preferably 20% by weight or less, relative to the total weight of the composition.

The amount of the hydrophobic surface-treated pigment(s) in the composition according to the present invention may be from 1% to 30% by weight, preferably from 5% to 25% by weight, and more preferably from 10% to 20% by weight, relative to the total weight of the composition.

### (Nonionic Surfactant)

The composition according to the present invention comprises at least one nonionic surfactant other than nonionic silicone surfactants. Two or more nonionic surfactants may be used in combination. Thus, a single type of the nonionic surfactant or a combination of different types of the nonionic surfactants may be used.

The nonionic surfactants are compounds well known in themselves (see, e.g., in this regard, “Handbook of Surfactants” by M. R. Porter, Blackie & Son publishers (Glasgow and London), 1991, pp. 116-178). Thus, they can, for example, be chosen from alcohols, alpha-diols, alkylphenols and esters of fatty acids, these compounds being ethoxylated, propoxylated or glycerolated and having at least one fatty chain comprising, for example, from 8 to 30 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils of plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives; amine oxides such as (C₁₀-C₁₄)alkylamine oxides or N—(C₁₀-C₁₄)acylaminopropylmorpholine oxides; silicone surfactants; and mixtures thereof.

The nonionic surfactants may preferably be chosen from monooxyalkylenated, polyoxyalkylenated, monoglycerolated or polyglycerolated nonionic surfactants. The oxyalkylene units are more particularly oxyethylene or oxypropylene units, or a combination thereof, and are preferably oxyethylene units.

Examples of monooxyalkylenated or polyoxyalkylenated nonionic surfactants that may be mentioned include:
monooxyalkylenated or polyoxyalkylenated (C₈-C₂₄)alkylphenols, saturated or unsaturated, linear or branched, monooxyalkylenated or polyoxyalkylenated C₈-C₃₀ alcohols,
saturated or unsaturated, linear or branched, monooxyalkylenated or polyoxyalkylenated C₈-C₃₀ amides,
esters of saturated or unsaturated, linear or branched, C₈-C₃₀ acids and of polyalkylene glycols,
monooxyalkylenated or polyoxyalkylenated esters of saturated or unsaturated, linear or branched, C₈-C₃₀ acids and of sorbitol,
saturated or unsaturated, monooxyalkylenated or polyoxyalkylenated plant oils, condensates of ethylene oxide and/or of propylene oxide, inter alia, alone or as mixtures.

The surfactants preferably contain a number of moles of ethylene oxide and/or of propylene oxide of between 1 and 100 and most preferably between 2 and 50. Advantageously, the nonionic surfactants do not comprise any oxypropylene units.

According to one of the embodiments of the present invention, the polyoxyalkylenated nonionic surfactants are chosen from polyoxyethylenated fatty alcohol (polyethylene glycol ether of fatty alcohol) and polyoxyethylenated fatty ester (polyethylene glycol ester of fatty acid).

Examples of polyoxyethylenated fatty alcohol (or C₈-C₃₀ alcohols) that may be mentioned include the adducts of ethylene oxide with lauryl alcohol, especially those containing from 7 to 50 oxyethylene units and more particularly those containing from 6 to 12 oxyethylene units (Laureth-6 to Laureth-12, as the CTFA names); the adducts of ethylene oxide with behenyl alcohol, especially those containing from 5 to 50 oxyethylene units (Beheneth-5 to Beheneth-50, as the CTFA names); the adducts of ethylene oxide with cetearyl alcohol (mixture of cetyl alcohol and stearyl alcohol), especially those containing from 7 to 30 oxyethylene units (Ceteareth-7 to Ceteareth-30, as the CTFA names); the adducts of ethylene oxide with cetyl alcohol, especially those containing from 7 to 30 oxyethylene units (Ceteth-7 to Ceteth-30, as the CTFA names); the adducts of ethylene oxide with stearyl alcohol, especially those containing from 7 to 30 oxyethylene units (Steareth-7 to Steareth-30, as the CTFA names); the adducts of ethylene oxide with isostearyl alcohol, especially those containing from 8 to 50 oxyethylene units (Isosteareth-8 to Isosteareth-50, as the CTFA names); and mixtures thereof.

As examples of monoglycerolated or polyglycerolated nonionic surfactants, monoglycerolated or polyglycerolated C₈-C₄₀ alcohols are preferably used.

In particular, the monoglycerolated or polyglycerolated C₈-C₄₀ alcohols correspond to the following formula:

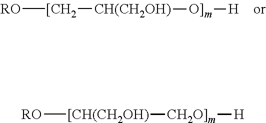

in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the present invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

Among the monoglycerolated or polyglycerolated alcohols, it is preferable to use the C₈/C₁ alcohol containing 1 mol of glycerol, the C₁₀/C₁₂ alcohol containing 1 mol of glycerol and the C₁₂ alcohol containing 1.5 mol of glycerol.

The monoglycerolated or polyglycerolated C₈-C₄₀ fatty esters may correspond to the following formula:

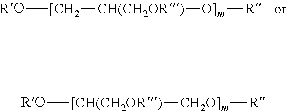

in which each of R′, R″ and R′″ independently represents a hydrogen atom, or a linear or branched C₅-C₄₀ and preferably C₈-C₃₀ alkyl-CO— or alkenyl-CO-radical, with the proviso that at least one of R′, R″ and R′″ is not a hydrogen atom, and m represents a number ranging from 1 to 30 and preferably from 1.5 to 10.

Examples of polyoxyethylenated fatty esters that may be mentioned include the adducts of ethylene oxide with esters of lauric acid, palmitic acid, stearic acid or behenic acid, and mixtures thereof, especially those containing from 9 to 100 oxyethylene units, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

According to one of the embodiments according to the present invention, the nonionic surfactant may be selected from esters of polyols with fatty acids with a saturated or unsaturated chain containing for example from 8 to 24 carbon atoms, preferably 12 to 22 carbon atoms, and polyoxyalkylenated derivatives thereof, preferably containing from 10 to 200, and more preferably from 10 to 100 oxyalkylene units, such as mono glyceryl esters or poly glyceryl esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; sorbitol esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; sugar (sucrose, maltose, glucose, fructose, and/or alkylglycose) esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; ethers of fatty alcohols; ethers of sugar and a C₈-C₂₄, preferably C₁₂-C₂₂, fatty alcohol or alcohols; and mixtures thereof.

As mono glyceryl esters of fatty acids, preferably C₁₂-C₂₂ fatty acids, glyceryl stearate (glyceryl mono-, di- and/or tristearate) (CTFA name: glyceryl stearate) or glyceryl ricinoleate and mixtures thereof can be cited, and as polyoxyalkylenated derivatives thereof, mono-, di- or triester of fatty acids with a polyoxyalkylenated glycerol (mono-, di- or triester of fatty acids with a polyalkylene glycol ether of glycerol), preferably polyoxyethylenated glyceryl stearate (mono-, di- and/or tristearate), such as PEG-20 glyceryl stearate (mono-, di-, tristearate and/or triisostearate) can be cited. Preferably, the polyoxyalkylenated derivative of mono glyceryl ester of fatty acids includes 10 to 40 oxyethylene units, such as PEG-20 glyceryl triisostearate.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEGIN by Goldschmidt (CTFA name: glyceryl stearate SE), can also be used.

As polyglyceryl esters of (a) fatty acid(s), preferably C₈-C₂₂ fatty acids, more preferably C₁₀-C₂₀ fatty acids (mono-, di- or triester of fatty acids with a polyglycerol) mention be made of the product containing 2 to 10 glycerol units, preferably 2 to 6 glycerol units, such as polyglyceryl laurate, oleate, myristate, caprylate, or stearate comprising 2 to 10 glycerol units, for example, polyglyceryl monolaurate comprising 2 to 10 glycerol units, polyglyceryl mono(iso)stearate comprising 2 to 10 glycerol units, polyglyceryl dioleate comprising 2 to 10 glycerol units, polyglyceryl dilaurate comprising 2 to 10 glycerol units, polyglyceryl dimyristate comprising 2 to 10 glycerol units, polyglyceryl trimyristate comprising 2 to 10 glycerol units, polyglyceryl trioleate comprising 2 to 10 glycerol units, and polyglyceryl tricaprylate comprising 2 to 10 glycerol units.

The sorbitol esters of C₈-C₂₄ fatty acids and polyoxyalkylenated derivatives thereof can be selected from sorbitan palmitate, sorbitan isostearate, sorbitan trioleate and esters of fatty acids and alkoxylated sorbitan containing for example from 20 to 100 EO, such as for example sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65, polyethylene sorbitan trioleate (polysorbate 85) or the compounds marketed under the trade names Tween 20 or Tween 60 by Uniqema.

As esters of fatty acids and glucose or alkylglucose, glucose palmitate, alkylglucose sesquistearates such as methylglucose sesquistearate, alkylglucose palmitates such as methylglucose or ethylglucose palmitate, methylglucoside fatty esters, the diester of methylglucoside and oleic acid (CTFA name: Methyl glucose dioleate), the mixed ester of methylglucoside and the mixture of oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Grillocose PSE-20 by GOLDSCHMIDT, and mixtures thereof, can for example be cited.

As sucrose esters, saccharose palmito-stearate, saccharose stearate and saccharose monolaurate can for example be cited.

As sugar ethers, alkylpolyglucosides can be used, and for example, ethers of a sugar and of C₈-C₂₄ fatty alcohols including decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and mixtures thereof can in particular be cited.

Mixtures of glycerides of alkoxylated plant oils such as mixtures of ethoxylated (200 EO) palm and copra (7 EO) glycerides can also be cited.

The nonionic surfactant according to the present invention preferably contains alkenyl or a branched C₁₂-C₂₂ acyl chain such as an oleyl or isostearyl group.

According to one of the embodiments according to the present invention, the nonionic surfactant may be selected from copolymers of ethylene oxide and of propylene oxide, in particular copolymers of the following formula:

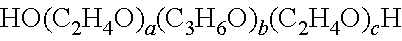

in which a, b and c are integers such that a+c ranges from 2 to 100 and b ranges from 14 to 60, and mixtures thereof.

The nonionic surfactant of the present invention is preferably selected from esters of polyols with fatty acids.

In a preferred embodiment, the nonionic surfactant of the present invention is preferably selected from mono glyceryl esters or poly glyceryl esters of a C₈-C₂₄, preferably C₁₂-C₂₂ fatty acid. The fatty acid may be saturated or unsaturated, preferably saturated and linear or branched, preferably branched. More preferably, the fatty acid is saturated and branched.

In another preferred embodiment of the present invention, the polyol of the nonionic surfactant comprises at least one sugar alcohol. The sugar alcohol may be selected from mannitol, erythritol, xylitol, sorbitol, arabitol, pentaerythritol, and mixtures thereof. Preferably, the sugar alcohol is sorbitol.

In a further preferred embodiment of the present invention, the nonionic surfactant is selected from sorbitol esters of a C₈-C₂₄, preferably C₁₂-C₂₂, fatty acid. More preferably, the nonionic surfactant is selected from sorbitol esters of a saturated and branched C₈-C₂₄, preferably C₁₂-C₂₂, fatty acid or acids.

The sorbitol esters of C₈-C₂₄ fatty acids and polyoxyalkylenated derivatives thereof can be selected from sorbitan palmitate, sorbitan isostearate, sorbitan trioleate and esters of fatty acids.

In one specific embodiment of the preset invention, the nonionic surfactant of the present invention does not include polyoxyalkylene units.

The nonionic surfactant(s) may be present in an amount of 0.1% by weight or more, preferably 0.3% by weight or more, and more preferably 0.5% by weight or more, and/or may be present in an amount of 10% by weight or less, preferably 5% by weight or less, and more preferably 3% by weight or less, relative to the total weight of the composition.

The amount of the nonionic surfactant(s) in the composition according to the present invention may be from 0.1% to 10% by weight, preferably from 0.3% to 5% by weight, and more preferably from 0.5% to 3% by weight, relative to the total weight of the composition.

### Aqueous Phase

The aqueous phase of the present invention comprises at least the (e) polyol, the (f) monoalcohol, and the (g) anionic polymer. The aqueous phase may include an aqueous medium, i.e., water and optionally a water-soluble solvent in addition to the ingredients (e) to (g).

The amount of the aqueous phase is at least 30% by weight relative to the total weight of the composition, and preferably at least 35% by weight relative to the total weight of the composition. The amount of the aqueous phase may be 80% by weight or less, preferably 60% by weight or less, more preferably 50% by weight or less, and even more preferably 40% by weight or less, relative to the total weight of the composition.

In the present invention, the term “water-soluble solvent” denotes a compound that is liquid at room temperature and water-miscible (miscibility with water of greater than 50% by weight at 25° C. and atmospheric pressure).

In one embodiment of the present invention, the amount of water and alcohol(s) including monoalcohol(s) and polyol(s) is at least 30% by weight, and preferably at least 35% by weight, relative to the total weight of the composition. The amount of the water and alcohol(s) including monoalcohol(s) and polyol(s) may be 70% by weight or less, preferably 60% by weight or less, more preferably 50% by weight or less, and even more preferably 40% by weight or less, relative to the total weight of the composition.

### (Polyol)

The composition according to the present invention comprises at least one polyol. Two or more polyols may be used in combination. Thus, a single type of the polyol or a combination of different types of the polyols may be used.

For the purposes of the present invention, the term “polyol” should be understood as meaning any organic molecule comprising at least two free hydroxyl groups.

The polyol that is suitable for use in the invention may be a compound of linear, branched or cyclic, saturated or unsaturated alkyl type, bearing at least two —OH functions on the alkyl chain.

Preferably, a polyol that may be used in the composition according to the invention is a compound of linear or branched, preferably linear alkyl type bearing at least two —OH functions, preferably 2 to 5 —OH functions, more preferably 2 to 4 —OH functions, and even more preferably 2 or 3 —OH functions on the alkyl chain.

The polyols that are advantageously suitable for formulating the cosmetic compositions according to the present invention are those especially having from 2 to 8 carbon atoms or for example, 3 to 6 carbon atoms.

The polyols that may be used according to the present invention are chosen from linear or branched, preferably linear polyols having from 3 to 8 carbon atoms; mention may be made especially of:
diols such as hexylene glycol, dipropylene glycol, pentylene glycol, propylene glycol and butylene glycol; and
triols, such as glycerol (glycerin),
   and mixtures thereof.

In one preferred embodiment of the present invention, the amount of polyol(s) in the composition is at least 6% by weight relative to the total weight of the composition. The polyol(s) may be present in an amount of 30% by weight or less, preferably 25% by weight or less, more preferably 20% by weight or less, and even more preferably 15% by weight or less, relative to the total weight of the composition.

In one preferred embodiment of the present invention, the polyol comprises at least one diol and at least one polyol having three or more —OH functions, in particular triol, in combination.

In this embodiment, the composition according to the present invention may comprise at least 3% by weight of the diol and at least 3% by weight of the polyol having three or more —OH functions in combination, relative to the total weight of the composition.

Specifically, the composition may comprise a combination of at least one diol and at least one triol. In this embodiment, the composition according to the present invention may comprise at least 3% by weight of the diol and at least 3% by weight of the triol in combination, relative to the total weight of the composition. In another embodiment, the composition according to the present invention comprises the diol in an amount range from 1% to 20% by weight and the triol in an amount ranging from 1% to 10% by weight; more preferably the diol in an amount ranging from 2% to 15% by weight and the triol in an amount ranging from 2% to 10% by weight; and even more preferably the diol in an amount ranging from 3% to 15% by weight and the triol in an amount ranging from 3% to 10% by weight relative to the total weight of the composition.

### (Monoalcohol)

The composition according to the present invention comprises at least one monoalcohol in the aqueous phase. Two or more types of monoalcohols may be used in combination. Thus, a single type of monoalcohol or a combination of different types of monoalcohols may be used.

The monoalcohol forms the aqueous phase of the present invention. Therefore, the monoalcohol here can mean a water-soluble, hydrophilic monoalcohol. For the purpose of the present invention, the term “hydrophilic” here means that a substance is soluble in water at a concentration of at least 1% by weight relative to the total weight of the water at room temperature (25° C.) and atmospheric pressure (105 Pa).

The monoalcohol may be linear or branched, saturated or unsaturated mono-alcohols having from 1 to 8 carbon atoms, preferably from 2 to 8 carbon atoms, bearing only one hydroxyl (OH) function.

In one embodiment, the monoalcohol may be an aliphatic monoalcohol having from 1 to 8 carbon atoms, preferably from 2 to 8 carbon atoms.

The term “aliphatic monoalcohol” here means any linear or branched, saturated alkane compound bearing only one hydroxyl (OH) function.

The aliphatic monoalcohol(s) present in the compositions of the invention may be chosen from ethanol, propanol, butanol, isopropanol, isobutanol and mixtures thereof.

In one preferred embodiment of the present invention, the monoalcohol can be selected from linear aliphatic monoalcohol having from 1 to 8 carbon atoms, preferably from 2 to 8 carbon atoms, such as ethanol, propanol, butanol, and mixtures thereof.

The amount of the monoalcohol in the composition according to the present invention may be 1% by weight or more, preferably 3% by weight or more, and more preferably 5% by weight or more; and/or may be 20% or less, preferably 15% by weight or less, and more preferably 10% by weight or less, relative to the total weight of the composition.

The amount of the monoalcohol in the composition according to the present invention may be from 1% to 20% by weight, preferably from 3% to 15% by weight, and more preferably from 5% to 10% by weight, relative to the total weight of the composition.

### (Anionic Polymer)

The composition according to the present invention comprises at least one anionic polymer. Two or more types of anionic polymer may be used in combination. Thus, a single type of the anionic polymer or a combination of different types of the anionic polymer may be used.

The anionic polymer is present in the aqueous phase of the present invention. Therefore, the anionic polymer here can be water-soluble, and hydrophilic. For the purpose of the present invention, the term “hydrophilic” here means that a substance is soluble in water at a concentration of at least 1% by weight relative to the total weight of the water at room temperature (25° C.) and atmospheric pressure (105 Pa).

An anionic polymer has a positive charge density. The charge density of the anionic polymer may be from 0.1 meq/g to 20 meq/g, preferably from 1 to 15 meq/g, and more preferably from 4 to 10 meq/g if the anionic polymer is a synthetic anionic polymer, and the average substitution degree of the anionic polymer may be from 0.1 to 3.0, preferably from 0.2 to 2.7, and more preferably from 0.3 to 2.5 if the anionic polymer is a natural anionic polymer.

It may be preferable that the molecular weight of the anionic polymer be 1,000 or more, preferably 5,000 or more, even more preferably 10,000 or more, even more preferably 15,000 or more, and in particular 20,000 or more; and/or be 1,000,000 or less, preferably 500,000 or less, more preferably 200,000 or less, and even more preferably 100,000 or less, an in particular 50,000 or less.

It may be preferable that the molecular weight of the anionic polymer ranges from 1,000 to 1,000,000, preferably from 5,000 to 500,000, even more preferably from 10,000 to 200,000, even more preferably from 15,000 to 100,000, and in particular from 20,000 to 50,000.

Unless otherwise defined in the descriptions, “molecular weight” may mean a number average molecular weight.

The anionic polymer may have at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a carboxylic group, and a carboxylate group.

The anionic polymer may be a homopolymer or a copolymer. The term “copolymer” is understood to mean both copolymers obtained from two kinds of monomers and those obtained from more than two kinds of monomers, such as terpolymers obtained from three kinds of monomers.

The anionic polymer may be selected from natural and synthetic anionic polymers.

The anionic polymer may comprise at least one hydrophobic chain.

The anionic polymer which may comprise at least one hydrophobic chain may be obtained by copolymerization of a monomer (a) chosen from carboxylic acids comprising an α,β-ethylenic unsaturation (monomer a′) and 2-acrylamido-2-methylpropanesulphonic acid (monomer a″) with a non-surface-active monomer (b) comprising an ethylenic unsaturation other than (a) and/or a monomer (c) comprising an ethylenic unsaturation resulting from the reaction of an acrylic monomer comprising an α,β-monoethylenic unsaturation or of an isocyanate monomer comprising a monoethylenic unsaturation with a monohydric nonionic amphiphilic component or with a primary or secondary fatty amine.

Thus, the anionic polymer with at least one hydrophobic chain may be obtained by two synthetic routes:
either by copolymerization of the monomers (a′) and (c), or (a′), (b) and (c), or (a″) and (c), or (a″), (b) and (c),
or by modification (and in particular esterification or amidation) of a copolymer formed from the monomers (a′) or from the monomers (a′) and (b), or (a″) and (b), by a monohydric nonionic amphiphilic compound or a primary or secondary fatty amine.

Mention may in particular be made, as 2-acrylamido-2-methylpropanesulphonic acid copolymers, of those disclosed in the article “Micelle formation of random copolymers of sodium 2-(acrylamido)-2-methylpropanesulfonate and nonionic surfactant macromonomer in water as studied by fluorescence and dynamic light scattering—Macromolecules, 2000, Vol. 33, No. 10-3694-3704” and in applications EP-A-0 750 899 and EP-A-1 069 172.

The carboxylic acid comprising an α,β-monoethylenic unsaturation constituting the monomer (a′) can be chosen from numerous acids and in particular from acrylic acid, methacrylic acid, crotonic acid, itaconic acid and maleic acid. It is preferably acrylic or methacrylic acid.

The copolymer can comprise a monomer (b) comprising a monoethylenic unsaturation which does not have a surfactant property. The preferred monomers are those which give water-insoluble polymers when they are homopolymerized. They can be chosen, for example, from C₁-C₄ alkyl acrylates and methacrylates, such as methyl acrylate, ethyl acrylate, butyl acrylate or the corresponding methacrylates. The more particularly preferred monomers are methyl acrylate and ethyl acrylate. The other monomers which can be used are, for example, styrene, vinyltoluene, vinyl acetate, acrylonitrile and vinylidene chloride. Unreactive monomers are preferred, these monomers being those in which the single ethylenic group is the only group which is reactive under the polymerization conditions. However, monomers which comprise groups which react under the effect of heat, such as hydroxyethyl acrylate, can optionally be used.

The monomer (c) is obtained by reaction of an acrylic monomer comprising α,β-monoethylenic unsaturation, such as (a), or of an isocyanate monomer comprising monoethylenic unsaturation with a monohydric nonionic amphiphilic compound or a primary or secondary fatty amine.

The monohydric nonionic amphiphilic compounds or the primary or secondary fatty amines used to produce the nonionic monomer (c) are well known. The monohydric nonionic amphiphilic compounds are generally alkoxylated hydrophobic compounds comprising an alkylene oxide forming the hydrophilic part of the molecule. The hydrophobic compounds are generally composed of an aliphatic alcohol or an alkylphenol, in which compounds a carbonaceous chain comprising at least six carbon atoms constitutes the hydrophobic part of the amphiphilic compound.

The preferred monohydric nonionic amphiphilic compounds are compounds having the following formula (V):

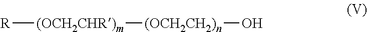

in which R is chosen from alkyl or alkylene groups comprising from 6 to 30 carbon atoms and alkylaryl groups having alkyl radicals comprising from 8 to 30 carbon atoms, R′ is chosen from alkyl groups comprising from 1 to 4 carbon atoms, n is a mean number ranging from approximately 1 to 150 and m is a mean number ranging from approximately 0 to 50, provided that n is at least as great as m.

Preferably, in the compounds of formula (V), the R group is chosen from alkyl groups comprising from 12 to 26 carbon atoms and alkylphenyl groups in which the alkyl group is C₈-C₁₃; the R′ group is the methyl group; m=0 and n=1 to 25.

The preferred primary and secondary fatty amines are composed of one or two alkyl chains comprising from 6 to 30 carbon atoms.

The monomer used to form the nonionic urethane monomer (c) can be chosen from highly varied compounds. Use may be made of any compound comprising a copolymerizable unsaturation, such as an acrylic, methacrylic or allylic unsaturation. The monomer (c) can be obtained in particular from an isocyanate comprising a monoethylenic unsaturation, such as, in particular, α,α-dimethyl-m-isopropenylbenzyl isocyanate.

The monomer (c) can be chosen in particular from acrylates, methacrylates or itaconates of oxyethylenated (1 to 50 EO) C₆-C₃₀ fatty alcohol, such as steareth-20 methacrylate, oxyethylenated (25 EO) behenyl methacrylate, oxyethylenated (20 EO) monocetyl itaconate, oxyethylenated (20 EO) monostearyl itaconate or the acrylate modified by polyoxyethylenated (25 EO) C₁₂-C₂₄ alcohols and from dimethyl-m-isopropenylbenzyl isocyanates of oxyethylenated (1 to 50 EO) C₆-C₃₀ fatty alcohol, such as, in particular, the dimethyl-m-isopropenylbenzyl isocyanate of oxyethylenated behenyl alcohol.

According to a specific embodiment of the present invention, the anionic polymer is chosen from acrylic terpolymers obtained from (a) a carboxylic acid comprising an α,β-ethylenic unsaturation, (b) a non-surface-active monomer comprising an ethylenic unsaturation other than (a), and (c) a nonionic urethane monomer which is the reaction product of a monohydric nonionic amphiphilic compound with an isocyanate comprising a monoethylenic unsaturation.

Mention may in particular be made, as anionic polymers comprising at least one hydrophobic chain, of the acrylic acid/ethyl acrylate/alkyl acrylate terpolymer, such as the product as a 30% aqueous dispersion sold under the name Acusol 823 by Rohm & Haas; the acrylates/steareth-20 methacrylate copolymer, such as the product sold under the name Aculyn 22 by Rohm & Haas; the (meth)acrylic acid/ethyl acrylate/oxyethylenated (25 EO) behenyl methacrylate terpolymer, such as the product as an aqueous emulsion sold under the name Aculyn 28 by Rohm & Haas; the acrylic acid/oxyethylenated (20 EO) monocetyl itaconate copolymer, such as the product as a 30% aqueous dispersion sold under the name Structure 3001 by National Starch; the acrylic acid/oxyethylenated (20 EO) monostearyl itaconate copolymer, such as the product as a 30% aqueous dispersion sold under the name Structure 2001 by National Starch; the acrylates/acrylate modified by polyoxyethylenated (25 EO) C₁₂-C₂₄ alcohol copolymer, such as the 30-32% copolymer latex sold under the name Synthalen W2000 by 3V SA; or the methacrylic acid/methyl acrylate/dimethyl-meta-isopropenylbenzyl isocyanate of ethoxylated behenyl alcohol terpolymer, such as the product as a 24% aqueous dispersion and comprising 40 ethylene oxide groups disclosed in the document EP-A-0 173 109.

The anionic polymers may also be Polyester-5, such as the product sold under the name of Eastman AQ™ 55S Polymer by EASTMAN CHEMICAL having the chemical formula below.

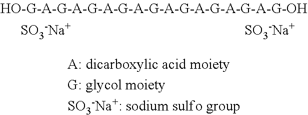

OH: hydroxyl group

It may be preferable that the anionic polymer be selected from the group consisting of polysaccharides such as alginic acid, hyaluronic acid, and cellulose polymers (e.g., carboxymethylcellulose), anionic (co)polyaminoacids such as (co)polyglutamic acids, (co)poly(meth)acrylic acids, (co)polyamic acids, (co)polystyrene sulfonate, (co)poly(vinyl sulfate), dextran sulfate, chondroitin sulfate, (co)polymaleic acids, (co)polyfumaric acids, maleic acid (co)polymers, and salts thereof.

The maleic acid copolymer may comprise one or more maleic acid comonomers, and one or more comonomers chosen from vinyl acetate, vinyl alcohol, vinylpyrrolidone, olefins comprising from 2 to 20 carbon atoms, and styrene.

Thus, the “maleic acid copolymer” is understood to mean any polymer obtained by copolymerization of one or more maleic acid comonomers and of one or more comonomers chosen from vinyl acetate, vinyl alcohol, vinylpyrrolidone, olefins comprising from 2 to 20 carbon atoms, such as octadecene, ethylene, isobutylene, diisobutylene or isooctylene, and styrene, the maleic acid comonomers optionally being partially or completely hydrolysed. Use will preferably be made of hydrophilic polymers, that is to say polymers having a solubility in water of greater than or equal to 2 g/l.

In an advantageous aspect of the present invention, the maleic acid copolymer may have a molar fraction of maleic acid units of between 0.1 and 1, more preferably between 0.4 and 0.9.

The weight-average molar mass of the maleic acid copolymer may be between 1,000 and 500,000, and preferably between 1,000 and 50,000.

It is preferable that the maleic acid copolymer be a styrene/maleic acid copolymer, and more preferably sodium styrene/maleic acid copolymer.

Use will preferably be made of a copolymer of styrene and of maleic acid in a 50/50 ratio.

Use may be made, for example, of the styrene/maleic acid (50/50) copolymer, in the form of an ammonium salt at 30% in water, sold under the reference SMA1000H® by Cray Valley or the styrene/maleic acid (50/50) copolymer, in the form of a sodium salt at 40% in water, sold under the reference SMA1000HNa® by Cray Valley.

The use of the styrene/maleic acid copolymer such as sodium styrene/maleic acid copolymer may improve the wettability of a film prepared by the composition according to the present invention.

According to one embodiment of the present invention, it is preferable that the anionic polymer be selected from hyaluronic acid and derivatives thereof.

Hyaluronic acid can be represented by the following chemical formula.

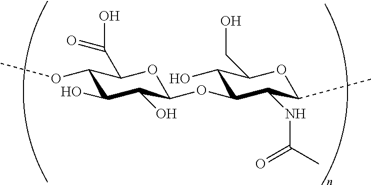

In the context of the present invention, the term “hyaluronic acid” covers in particular the basic unit of hyaluronic acid of formula:

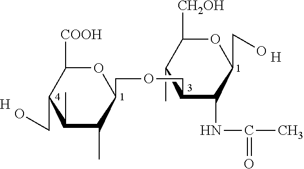

This is the smallest fraction of hyaluronic acid comprising a disaccharide dimer, namely D-glucuronic acid and N-acetylglucosamine.

The term “hyaluronic acid and derivatives thereof” also comprises, in the context of the present invention, the linear polymer comprising the polymeric unit described above, linked together in the chain via alternating β(1,4) and β(1,3) glycosidic linkages, having a molecular weight (MW) that can range between 380 and 1,000,000 daltons. This molecular weight depends in large part on the source from which the hyaluronic acid is obtained, and/or on the preparation methods.

The term “hyaluronic acid and derivatives thereof” also comprises, in the context of the present invention, hyaluronic acid salts. As the salts, mention may be made of alkaline metal salts such as sodium salts and potassium salts, alkaline earth metal salts such as magnesium salts, ammonium salts, and mixtures thereof.

In the natural state, hyaluronic acid is present in pericellular gels, in the base substance of the connective tissues of vertebrate organs such as the dermis and epithelial tissues, and in particular in the epidermis, in the synovial fluid of the joints, in the vitreous humor, in the human umbilical cord and in the crista gaili apophysis.

Thus, the term “hyaluronic acid and derivatives thereof” comprises all the fractions or subunits of hyaluronic acid having a molecular weight in particular within the molecular weight range recalled above.

In the context of the present invention, hyaluronic acid fractions which do not have inflammatory activity are preferably used.

By way of illustration of the various hyaluronic acid fractions, reference may be made to the document “Hyaluronan fragments: an information-rich system”, R. Stern et al., European Journal of Cell Biology 58 (2006) 699-715, which reviews the listed biological activities of hyaluronic acid according to its molecular weight.

According to a preferred embodiment of the present invention, the hyaluronic acid fractions suitable for the use covered by the present invention have a molecular weight of less than 50,000 Da, which is a so-called low-molecular-weight hyaluronic acid.

Finally, the term “hyaluronic acid and derivatives thereof” also comprises hyaluronic acid esters in particular those in which all or some of the carboxylic groups of the acid functions are esterified with oxyethylenated alkyls or alcohols, containing from 1 to 20 carbon atoms, in particular with a degree of substitution at the level of the D-glucuronic acid of the hyaluronic acid ranging from 0.5 to 50%.

Mention may in particular be made of methyl, ethyl, n-propyl, n-pentyl, benzyl and dodecyl esters of hyaluronic acid. Such esters have in particular been described in D. Campoccia et al. “Semisynthetic resorbable materials from hyaluronan esterification”, Biomaterials 19 (1998) 2101-2127.

The hyaluronic acid derivative may be, for example, acetylated hyaluronic acid or a salt thereof.

The molecular weights indicated above are also valid for the hyaluronic acid esters.

The amount of the anionic polymer(s) in the composition according to the present invention may be 0.01% by weight or more, preferably 0.05% by weight or more, and more preferably 0.1% by weight or more, relative to the total weight of the composition.

The amount of the anionic polymer(s) in the composition according to the present invention may be 20% by weight or less, preferably 15% by weight or less, and more preferably 10% by weight or less, relative to the total weight of the composition.

The amount of the anionic polymer(s) in the composition according to the present invention may be from 0.01% to 20% by weight, preferably from 0.05% to 15% by weight, and more preferably from 0.1% to 10% by weight, relative to the total weight of the composition.

### (Other Ingredients)

### Oil

The composition according to the present invention may comprise at least one oil in the oily phase. Two or more types of oils may be used in combination. Thus, a single type of oil or a combination of different types of oils may be used.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination. These oils may be volatile or non-volatile.

Among the oils which may be used in the present invention, mention may be made of: volatile or non-volatile oils; these oils may be hydrocarbon-based oils, especially of animal or plant origin, synthetic oils, silicone oils, fluoro oils, or mixtures thereof.

For the purposes of the present invention, “hydrocarbon-based oil” or “hydrocarbon oil” is intended to mean an oil mainly containing hydrogen and carbon atoms and optionally oxygen, nitrogen, sulfur and/or phosphorus atoms. The hydrocarbon-based oil does not comprise any silicon atoms.

For the purposes of the present invention, “silicone oil” is intended to mean an oil comprising at least one silicon atom, and especially at least one Si—O group.

For the purposes of the present invention, “polar oil” is intended to mean an oil of which the solubility parameter 6a at 25° C. is other than 0 (J/cm³)^{1/2}.

In particular, “polar oil” is intended to mean an oil of which the chemical structure is formed essentially from, or even constituted of, carbon and hydrogen atoms, and comprising at least one highly electronegative heteroatom such as an oxygen, nitrogen, silicon or phosphorus atom.

The definition and calculation of the solubility parameters in the Hansen three-dimensional solubility space are described in the article by C. M. Hansen: The three-dimensional solubility parameters, J. Paint Technol., 39, 105 (1967).

According to this Hansen space:
δ_{D} characterizes the London dispersion forces resulting from the formation of dipoles induced during molecular impacts;
δ_{P} characterizes the Debye interaction forces between permanent dipoles and also the Keesom interaction forces between induced dipoles and permanent dipoles;
δₕ characterizes the forces of specific interactions (such as hydrogen bonds, acid/base bonds, donor/acceptor bonds, and the like);
δₐ is determined by the equation: βₐ=(δₚ²+δₕ²)^{1/2}.

The parameters δₚ, δₕ, δ_{D} and δₐ are expressed as (J/cm³)^{1/2}.

Preferably, the polar oils used according to the present invention have a δₐ of between 4 and 9.1, preferably a 8a of between 6 and 9.1, even better still between 7.3 and 9.1.

The oil may be a non-polar oil such as a hydrocarbon oil, a silicone oil, or the like; a polar oil such as a plant or animal oil and an ester oil or an ether oil; or a mixture thereof.

The oil may be selected from the group consisting of oils of plant or animal origin, synthetic oils, silicone oils, hydrocarbon oils, and fatty alcohols.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils apart from the alkyl or alkylene carbonate, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived, is branched.

The ester oils of the monoesters of monoacids and of monoalcohols may be represented by formula R₁COOR₂ in which R₁ represents the residue of a linear or branched, preferably a linear fatty acid comprising from 1 to 40 carbon atoms, preferably 6 to 24 carbon atoms, and more preferably 10 to 20 carbon atoms, and R₂ represents a hydrocarbon-based chain, especially branched, containing from 1 to 40 carbon atoms, preferably 1 to 12 carbon atoms, and more preferable 2 to 8 carbon atoms, with the proviso that R₁+R₂ is ≥10.

Among the monoesters of monoacids and of monoalcohols, mention may be made of ethyl palmitate, ethyl hexyl palmitate, isopropyl palmitate, alkyl myristates such as isopropyl myristate or ethyl myristate, isocetyl stearate, 2-ethylhexyl isononanoate, isononyl isononanoate, isodecyl neopentanoate, and isostearyl neopentanoate.

It is preferred that the ester oil is selected from fatty acid ester oils.

Esters of C₄-C₂₂ dicarboxylic or tricarboxylic acids and of C₁-C₂₂ alcohols, and esters of monocarboxylic, dicarboxylic, or tricarboxylic acids and of non-sugar C₄-C₂₆ dihydroxy, trihydroxy, tetrahydroxy, or pentahydroxy alcohols may also be used.

Mention may especially be made of: diethyl sebacate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides, or polysaccharides.

Examples of suitable sugars that may be mentioned include sucrose (or saccharose), glucose, galactose, ribose, fucose, maltose, fructose, mannose, arabinose, xylose, and lactose, and derivatives thereof, especially alkyl derivatives, such as methyl derivatives, for instance, methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters, and polyesters, and mixtures thereof.

These esters may be, for example, oleates, laurates, palmitates, myristates, behenates, cocoates, stearates, linoleates, linolenates, caprates, and arachidonates, or mixtures thereof such as, especially, oleopalmitate, oleostearate, and palmitostearate mixed esters, as well as pentaerythrityl tetraethyl hexanoate.

More particularly, use is made of monoesters and diesters and especially sucrose, glucose, or methylglucose monooleates or dioleates, stearates, behenates, oleopalmitates, linoleates, linolenates, and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As the ether oil, dialkyl ethers such as those represented by the following formula:

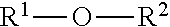

wherein
each of R¹ and R² independently denotes a linear, branched or cyclic C₄-C₂₄ alkyl group, preferably C₆-C₁₈ alkyl group, and more preferably C₈-C₁₂ alkyl group. It is preferable that R¹ and R² are the same.

As the linear alkyl group, mention may be made of a butyl group, a pentyl group, a hexyl group, a heptyl group, an octyl group, a nonyl group, a decyl group, an undecyl group, a dodecyl group, tridecyl group, a tetradecyl group, a pentadecyl group, a hexadecyl group, a heptadecyl group, an octadecyl group, a nonadecyl group, an eicosyl group, a behenyl group, a docosyl group, a tricosyl group, and a tetracosyl group.

As the branched alkyl group, mention may be made of a 1-methylpropyl group, 2-methylpropyl group, a t-butyl group, a 1,1-dimethylpropyl group, a 3-methylhexyl group, a 5-methylhexyl group, an 1-ethylhexyl group, an 2-ethylhexylgroup, a 1-butylpentyl group, a 5-methyloctyl group, an 1-ethylhexyl group, an 2-ethylhexyl group, a 1-butylpentyl group, a 5-methyloctyl group, a 2-butyloctyl group, an isotridecyl group, a 2-pentylnonyl group, a 2-hexyldecyl group, an isostearyl group, a 2-heptylundecyl group, an 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyyl group, and a 2-(1,3,3-trimethylbutyl)-5,7,7-trimethyloctyl group.

As the cyclic alkyl group, mention may be made of a cyclohexyl group, a 3-methylcyclohexyl group, and a 3,3,5-trimethylcyclohexyl group.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate), and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oil is chosen from liquid polydialkylsiloxanes or dimethicone, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of the formula:

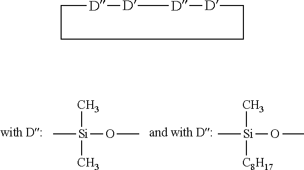

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

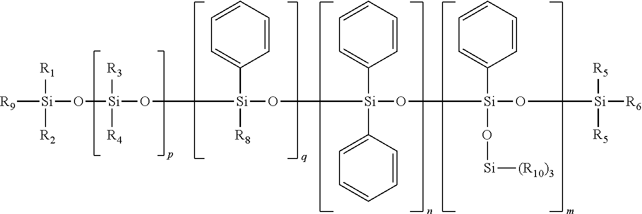

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl, or butyl radicals, and
m, n, p, and q are, independently of each other, integers of 0 to 900 inclusive, preferably 0 to 500 inclusive, and more preferably 0 to 100 inclusive, with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250, and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

The hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane, and isodecane;
linear or branched hydrocarbons containing more than 16 carbon atoms, such as liquid paraffins, liquid petroleum jelly, polydecenes and hydrogenated polyisobutenes such as Parleam®, and squalane; and
mixtures of alkanes, for example, C9-12 Alkane, C10-13 Alkane, C13-14 Alkane, C13-15 Alkane, C14-17 Alkane, C14-19 Alkane, C15-19 Alkane, C15-23 Alkane, C18-21 Alkane, C8-9 Alkane/Cycloalkane, C9-10 Alkane/Cycloalkane, C9-11 Alkane/Cycloalkane, C9-16 Alkane/Cycloalkane, C10-12 Alkane/Cycloalkane, C11-14 Alkane/Cycloalkane, C11-Alkane/Cycloalkane, C12-13 Alkane/Cycloalkane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 8 or more, preferably 10 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 8 to 40 carbon atoms, preferably from 10 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₈-C₃₀ alcohols, preferably straight or branched, saturated C₁₀-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₈-C₃₀ fatty alcohols. Among the linear or branched, saturated C₈-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from cetyl alcohol, octyldodecanol, hexyldecanol, isostearyl alcohol, and mixtures thereof.

It is also preferable that the oil be chosen from oils with a molecular weight below 600 g/mol.

Preferably, the oil has a low molecular weight such as below 600 g/mol, chosen among ester oils with a short hydrocarbon chain or chains (C₁-C₁₂) (e.g., isopropyl myristate, isopropyl palmitate, isononyl isononanoate, and ethyl hexyl palmitate), silicone oils (e.g., volatile silicones such as cyclohexasiloxane), hydrocarbon oils (e.g., isododecane, isohexadecane, and squalane), branched and/or unsaturated fatty alcohol (C₁₂-C₃₀) type oils such as octyldodecanol and oleyl alcohol, and ether oils such as dicaprylyl ether.

It is preferable that the oil be chosen from polar oils, and more preferably from ester oils, fatty alcohols, and a combination thereof. It is further preferred that the oil comprise both of ester oils and fatty alcohols, in particular the monoesters of monoacids and of monoalcohols represented by the formula R₁COOR₂ in which R₁ represents the residue of a linear fatty acid comprising from 10 to 20 carbon atoms, and R₂ represents a branched hydrocarbon-based chain containing from 2 to 8 carbon atoms and the fatty alcohol having the structure R—OH wherein R is chosen from saturated branched radicals containing from 4 to 40 carbon atoms, preferably from 6 to 30 carbon atoms, and more preferably from 12 to 20 carbon atoms.

The amount of the oil(s) in the composition according to the present invention may be 5% by weight or more, preferably 10% by weight or more, and more preferably 15% by weight or more; and/or may be 50% by weight or less, preferably 40% by weight or less, and more preferably 35% by weight or less, relative to the total weight of the composition.

The amount of the oil(s) in the composition according to the present invention may be from 5% to 40% by weight, preferably from 10% to 40% by weight, and more preferably from 15% to 35% by weight, relative to the total weight of the composition.

### Water

The composition according to the present invention preferably includes water in the aqueous phase.

The amount of water in the composition according to the present invention may be 5% by weight or more, preferably 10% by weight or more, and more preferably 15% by weight or more; and/or may be 50% or less, preferably 40% by weight or less, and more preferably 30% by weight or less, relative to the total weight of the composition.

The amount of water in the composition according to the present invention may be from 5% to 50% by weight, preferably from 10% to 40% by weight, and more preferably from 15% to 30% by weight, relative to the total weight of the composition.

### Thickening Aid

The composition according to the present invention may comprise at least one thickening aid selected from polyoxyalkylenated silicone and alkyl or alkylene carbonate. Two or more thickening aids may be used in combination. Thus, a single type of the thickening aid or a combination of different types of the thickening aids may be used.

The thickening aid of the present invention may be included in the oily phase and is used to aid lipophilic or oil thickener such as the spherical hydrophobic silica aerogel so as to achieve a good stability.

The thickening aid may be selected from polyoxyalkylenated silicone and alkyl or alkylene carbonate.

### Polyoxyalkylenated Silicone

A single type of the polyoxyalkylenated silicone or a combination of different types of the polyoxyalkylenated silicone may be used in the composition according to the present invention.

In the present invention, the term “polyoxyalkylenated silicone” means any silicone comprising at least one oxyalkylenated unit.

The oxyalkylenated units in the polyoxyalkylenated silicone can be polyoxyalkylene groups alternating oxygen atoms and linear or branched C₂ to C₁₀ alkylene groups, preferably C₂ to C₆ alkylene groups, and more preferentially C₂ and/or C₃ (respectively ethylene and propylene) alkylene groups. The oxyalkylenated units can be represented by (—CₓH₂ₓO—)ₐ wherein x ranges from 2 to 6 and a is greater than or equal to 2. Preferably, the oxyalkylenated units are oxyethylene or oxypropylene units, or a combination thereof, and are preferably oxyethylene units.

The polyoxyalkylenated silicone preferably contain a number of moles of ethylene oxide and/or of propylene oxide of between 2 and 100, more preferably between 3 and 50, even more preferably between 4 and 40. In one embodiment of the present invention, the polyoxyalkylenated silicone does not comprise any oxypropylene units.

In one embodiment, the polyoxyalkylenated silicone comprises from 2 to 50 oxyethylene units, preferably from 3 to 40 oxyethylene units, more preferably from 4 to 30 oxyethylene units, and even more preferably from 5 to 20 oxyethylene units.

In one embodiment of the present invention, the polyoxyalkylenated silicone includes dimethicone copolyols which are polyorganosiloxanes comprising oxyalkylenated units, such as polyethyleneoxy and/or polypropyleneoxy moieties.

The polyoxyalkylenated units of the polyoxyalkylenated silicone can be substituted with at least one alkyl group and/or amine group. The alkyl group may be a linear or branched C₁ to C₃₀ chain, preferably C₄ to C₂₂ chain. The substituted amine moieties may be chosen, for example, from amino C₁-C₄ alkyl moieties.

The polyoxyalkylenated silicone may be a compound of formula (I):

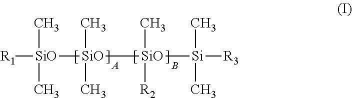

in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; with the proviso that A and B are not simultaneously equal to zero;
x is an integer ranging from 1 to 6;
y is an integer ranging from 1 to 30;
z is an integer ranging from 0 to 5.

According to one embodiment of the present invention, in the compound of formula (I), the alkyl radical is a methyl radical, x is an integer ranging from 2 to 6 and y is an integer ranging from 4 to 30.

As examples of polyoxyalkylenated silicone of formula (I), mention may be made of the compounds of formula (II):

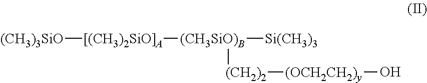

in which A is an integer ranging from 20 to 105, B is an integer ranging from 2 to 10 and y is an integer ranging from 10 to 20.

As examples of the polyoxyalkylenated silicone of formula (I), mention may also be made of the compounds of formula (III):

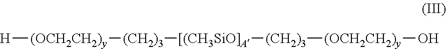

in which A′ and y are integers ranging from 10 to 20.

The polyoxyalkylenated silicone of the present invention which may be used are those sold by the company Dow Corning under the names DC 5329, DC 7439-146, DC 2-5695 and Q4-3667. The compounds DC 5329, DC 7439-146 and DC 2-5695 are compounds of formula (III) in which, respectively, A is 22, B is 2 and y is 12; A is 103, B is 10 and y is 12; A is 27, B is 3 and y is 12. The compound Q4-3667 is a compound of formula (III) in which A is 15 and y is 13.

Commercially available products of dimethicone copolyols include, for example, dimethicone copolyol as such as PEG10 dimethicone and PEG14 dimethicone; PEG12 dimethicone marked under the trade name XIAMETER® OFX-0193 FLUID by DOW CORNING, PEG/PPG 18/18 dimethicone silicones sold under the name 5225C by Dow Corning, Cetyl PEG/PPG-10/1 dimethicone silicones such as proposed under the name ABIL EM 90 by Evonik.

### Alkyl or Alkylene Carbonate

A single type of the alkyl or alkylene carbonate or a combination of different types of the alkyl or alkylene carbonate may be used in the composition according to the present invention.

According to one embodiment, the alkylene chain(s) of the alkylene carbonate(s) and/or the alkyl radical(s) of the alkyl carbonate(s) present in the composition according to the invention comprise(s) from 1 to 6 carbon atoms, preferably from 2 to 6 carbon atoms, and more preferably from 2 to 4 carbon atoms, and are eventually substituted by one or more hydroxyl groups.

According to another embodiment, the sum of the carbons of the alkylene chain(s) of alkylene carbonate(s) and/or the sum of the carbons of the alkyl group(s) of the alkyl carbonate(s) present in the composition according to the invention is (are) ranging from 2 to 6 carbon atoms.

The alkylene carbonates are notably chosen from those of formula (4) below:

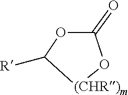

in which formula (4)
R′ denotes a hydrogen atom, a linear or branched C₁-C₆ alkyl radical, a linear or branched C₁-C₄ hydroxyalkyl radical;
R″ represents a hydrogen atom, a linear or branched C₁-C₆ alkyl radical, a linear or branched C₁-C₄ hydroxyalkyl radical;
m is 1, 2 or 3.

Preferably, the radical R′ represents a hydrogen atom, a linear or branched C₁-C₄ alkyl radical, a linear or branched C₁-C₂ hydroxyalkyl radical.

R″ represents a hydrogen atom, a linear or branched C₁-C₂ alkyl radical, a linear or branched C₁-C₂ hydroxyalkyl radical.

Preferably, m is 1.

As particularly advantageous examples of alkylene carbonates, mention may be made of the compounds for which the radical R′ represents a hydrogen atom (corresponding to ethylene carbonate), a methyl group (corresponding to propylene carbonate), ethyl (corresponding to 1,2-butylene carbonate), hydroxymethyl (R′═—CH₂OH; corresponding to glyceryl carbonate). Preferably, the alkylene carbonate used is propylene carbonate.

The alkyl carbonates are notably chosen from those of formula (5) below:

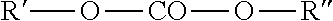

in which formula (5)
R′ denotes a linear or branched C₁-C₅ alkyl radical, a linear or branched C₁-C₄ hydroxyalkyl radical;
R″ represents a linear or branched C₁-C₅ alkyl radical, a linear or branched C₁-C₄ hydroxyalkyl radical;
the sum of the carbons of R′ and R″ ranging from 2 to 6.

Preferably, the radical R′ represents a linear C₁-C₃ alkyl radical or a linear C₁-C₂ hydroxyalkyl radical.

R″ represents a linear C₁-C₃ alkyl radical or a linear C₁-C₂ hydroxyalkyl radical.

More particularly, mention may be made of diethyl carbonate and dipropyl carbonate.

The carbonates according to the invention are preferably alkylene carbonates and more particularly propylene carbonate.

The thickening aid(s) may be present in an amount of 0.1% by weight or more, preferably 0.5% by weight or more, and more preferably 1% by weight or more, and/or may be present in an amount of 10% by weight or less, preferably 5% by weight or less, and more preferably 3% by weight or less, relative to the total weight of the composition.

The amount of the thickening aid(s) in the composition according to the present invention may be from 0.1% to 10% by weight, preferably from 0.5% to 5% by weight, and more preferably from 1% to 3% by weight, relative to the total weight of the composition.

### Film-Forming Polymer

The composition according to the present invention may comprise at least one film-forming polymer. Two or more film-forming polymers may be used in combination. Thus, a single type of film-forming polymer or a combination of different types of film-forming polymers may be used.

For the purposes of the present invention, the term “polymer” means a compound corresponding to the repetition of one or more units (these units being derived from compounds known as monomers). This or these units(s) are repeated at least twice and preferably at least three times.

The term “film-forming polymer” means a polymer that is capable of forming, by itself or in the presence of an auxiliary film-forming agent, a macroscopically continuous film that adheres to a support, especially to keratin materials, preferably a cohesive film, and better still a film whose cohesion and mechanical properties are such that the said film may be isolable and manipulable in isolation, for example, when the said film is prepared by pouring onto a non-stick surface, for instance, a Teflon-coated or silicone-coated surface.

According to one embodiment of the present invention, the film-forming polymer may be selected from the group comprising:
film-forming polymers that are soluble in an organic solvent medium, in particular liposoluble polymers; this means that the polymer is soluble or miscible in the organic medium and will form a single homogeneous phase when it is incorporated into the medium;
film-forming polymers that are dispersible in an organic solvent medium; this means that the polymer forms an insoluble phase in the organic medium, the polymer remaining stable and/or compatible once incorporated into this medium. In particular, such polymers may be in the form of non-aqueous dispersions of polymer particles, preferably dispersions in silicone-based or hydrocarbon-based oils; in one embodiment, the non-aqueous dispersions of polymer comprise polymer particles stabilized on their surface with at least one stabilizer; these non-aqueous dispersions are often referred to as “NADs”; and
film-forming polymers in the form of aqueous dispersions of polymer particles; this means that the polymer forms an insoluble phase in water, the polymer remaining stable and/or compatible once incorporated into the water, the polymer particles possibly being stabilized at their surface with at least one stabilizer. These polymer particles are often referred to as “lattices”; in this case, the composition must comprise an aqueous phase.

Preferably, the film-forming polymer is selected from the group consisting of polyamide-silicone block polymers, block ethylenic polymers, vinyl polymers comprising at least one carboxiloxane dendrimer derivative, copolymers comprising carboxylate groups and polydimethylsilixane groups, silicone resins, lipodispersible polymers in the form of a non-aqueous dispersion of polymer particles, olefin copolymers selected from amorphous olefin copolymers and olefin copolymers with controlled and moderate crystallization, hydrocarbon-based resins having a number-average molecular weight of less than or equal to 10,000 g/ml, and a mixture thereof, more preferably from silicone resins.

The film-forming silicone resin may be any silicone resin which has film-forming properties.

According to one embodiment of the present invention, the film-forming silicone resin may be selected from silsesquioxane, siloxysilicate and a resin obtained by hydroxysilylation.

The nomenclature of the silicone resin is known in the art under the name of “MDTQ” nomenclature, by which a silicone resin is described according to the various repeating siloxane monomer moieties which constitute the polymer. Each letter of “MDTQ” corresponds to a different type of moiety.

The symbol “M” corresponds to the monofunctional moiety (CH₃)₃SiO_{1/2}. This moiety is regarded as monofunctional because the silicon atom shares only one oxygen for the formation of the chain. The “M” moiety can be represented by the following structure:

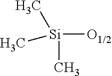

At least one of the methyl groups can be replaced so as, for example, to produce a moiety with the following formula: [R(CH₃)₂]SiO_{1/2}, such as represented by the following structure:

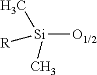

in which R is other than a methyl group.

The symbol “D” corresponds to the difunctional moiety (CH₃)SiO_{2/2} in which two of the available bonds on the silicon atom are used to bond with oxygen for the formation of the polymer chain. The “D” moiety, which is the essential component element of the dimethicone oils, can be represented by the following formula:

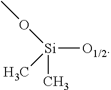

The symbol “T” corresponds to the trifunctional moiety (CH₃)SiO_{3/2}, in which three of the available bonds on the silicon atom are used to bond with oxygen for the formation of the polymer chain. The “T” moiety can be represented by the following structure:

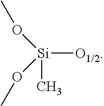

As in the “M” moiety, any one of the methyl groups can be replaced in “D” or “T” by an R group which is other than methyl.

Finally, the symbol “Q” corresponds to a quadrifunctional moiety SiO_{4/2}, in which all four available bonds on the silicon atom are used to bond with oxygen for the formation of the polymer chain. The “Q” moiety can be represented by the following structure:

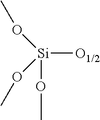

As described above, in one embodiment of the present invention, the film-forming silicone resin may be selected from the siloxysilicate, silsesquioxane and a resin obtained by hydrosilylation. Any siloxysilicate, silsesquioxane or resin obtained by hydroxysilylation, which acts as a film-forming agent, can be used in the composition of the present invention. The film-forming silicone resin preferably is crosslinked.

According to one embodiment of the present invention, the film-forming silicone resin may be selected from substituted siloxysilicate, silsesquioxane and resin obtained by hydroxysilylation. A substituted siloxysilicate or a substituted silsesquioxane may be, for example, a siloxysilicate or a silsesquioxane in which a methyl group has been replaced by a longer carbon chain, such as an ethane, propane or butane chain. The carbon chain may be saturated or nonsaturated.

According to one embodiment of the present invention, the film-forming silicone resin may be selected from siloxysilicate, such as MQ resins represented by the following formula:

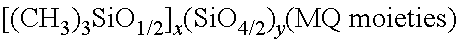

in which x and y may have values ranging from 20 to 100, preferably 50 to 80.

According to another embodiment of the present invention, siloxysilicate may be selected from all the combinations of M and of Q moieties such as, for example, [(R)₃Si]ₓ(SiO_{4/2})_{y}, in which R is selected from a methyl group and a longer carbon chain, such as C₂-C₁₀ alkyl groups.

According to another embodiment of the present invention, the film-forming silicone resin may be selected from silsesquioxane represented by the following formula:

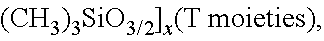

in which x has a value which can range up to several thousands and the CH₃ can be replaced by an R, such as described hereinabove for the T moieties.

Most preferably, the film-forming silicone resin is trimethylsiloxysilicate, for example, sold by the company Momentive Performance Materials under the name SR 1000 MQ Resin.

The film-forming polymer(s) may be present in an amount of 0.5% by weight or more, preferably 1% by weight or more, more preferably 3% by weight or more, and even more probably 5% by weight or more, and/or may be present in an amount of 20% by weight or less, preferably 15% by weight or less, more preferably 10% by weight or less, and most preferably 8% by weight or less, relative to the total weight of the composition.

The amount of the film-forming polymer(s) in the composition according to the present invention may be from 0.5% to 20% by weight, preferably from 1% to 15% by weight, more preferably from 3% to 10% by weight, and even more preferably 5% to 8% by weight relative to the total weight of the composition.

### Filler

The composition according to the present invention may comprise at least one filler other than the (a) spherical hydrophobic silica aerogel, the (b) at least one lipophilic thickener, and the (c) hydrophobic surface-treated pigment. Two or more fillers can be combined. The filler can be inorganic or organic, preferably inorganic.

As the inorganic filler mention may be made of talc, mica, silica, hollow silica, magnesium aluminum silicate, titanium dioxide, kaolin, bentone, calcium carbonate, magnesium hydrogen carbonate, hydroxyapatite, boron nitride, fluorphlogopite, sericite, calcinated talc, calcinated mica, calcinated sericite, synthetic mica, perlite, lauroyl lysine, metal soap, bismuth oxychloride, barium sulfate, magnesium sulfate, magnesium carbonate, and mixtures thereof, optionally hydrophilic- or hydrophobic-treated.

The inorganic filler may be composite silica particle. In the context of the present invention, the term “composite silica particles” means silica particles within which functional compounds, preferably metal oxides, are included. Therefore, preferably, the composite silica particles may refer to “metal oxide-including silica particles”. Most preferably, the metal oxides are scattered inside the silica particles.

The metal oxides may preferably be chosen from titanium oxide, zinc oxide, iron oxide and zirconium oxide, or mixtures thereof, and more particularly from titanium dioxide (TiO₂) and zinc oxide, and mixtures thereof. Particularly preferably, titanium dioxide may be used. In this embodiment, the composite silica particle is referred to Silica (and) Titanium Dioxide.

The composite silica particles may have an average particle size as determined by an image analysis method of 0.1 μm or more, preferably 0.5 μm or more, and more preferably 1 μm or more, and may have an average particle size by an image analysis method of 50 μm or less, preferably 20 μm or less, and more preferably 12 μm or less.

The “average particle size” can be determined in accordance with the following procedure: Particle sizes of 50 particles are measured using a SEM image and an average value of the particle sizes is calculated.

The composite silica particles may be porous or non-porous, and they may have a low oil-absorbing capacity.

In the composite silica particles, the weight ratio of silica to the functional compounds (preferably metal oxides and most preferably titanium dioxide) may be from 9:1 to 5:5, preferably from 4:1 to 3:2, and more preferably 7:3.

The composite silica particles may be surface-treated to be hydrophobic. For example, the composite silica particle may be surface-treated with alkylsilanes.

According to the present invention, the inorganic filler may have been surface-treated with least one silicone oil and/or at least one non-silicone oil.

As the organic filler, mention may be made of acrylic polymer powders, silicone powders, wax powders, polyamide powders, urethane polymer powders, tetrafluoroethylene polymer powders, polyacrylonitrile powders, poly-β-alanine powders, polyethylene powders, polytetrafluoroethylene powders, (meth)acrylic or (meth)acrylate powders, lauroyllysine, starch, cellulose powder, tetrafluoroethylene polymer powders and mixtures thereof.

The (meth)acrylic or (meth)acrylate powders can include, for example, polymethylmethacrylate crosspolymer, methyl methacrylate/glycol dimethacrylate crosspolymer, polymethyl methacrylate/ethylene glycol dimethacrylate powders, polyallyl methacrylate/ethylene glycol dimethacrylate powders, and ethylene glycol dimethacrylate/lauryl methacrylate copolymer powders.

As the polyamide powders, mention may be made of those sold under the name “Orgasol” by the company Atochem. These polyamide powder particles are moreover known according to their various physicochemical properties under the name “Nylon 12” or “Nylon 6”. The polyamide powders useful in the present invention may also include those sold under the name SP500 by the company TORAY.

The filler(s) may be present in the composition in a content of 1% by weight or more, preferably 5% by weight or more, and more preferably 10% by weight or more, and it may be present in the composition in a content of 30% by weight or less, preferably 25% by weight or less, and more preferably 20% by weight or less, relative to the total weight of the composition.

The amount of the filler(s) in the composition according to the present invention may be from 1% to 30% by weight, preferably from 5% to 25% by weight, and more preferably from 10% to 20% by weight, relative to the total weight of the composition.

### Skincare Active Agent

The composition according to the present invention may comprise at least one skincare active agent. If two or more skincare active agents are used, they may be the same or different.

It is preferable that the skin care active ingredient be a skin care cosmetic active ingredient, and more preferably a skin peeling agent, a skin whitening agent, or a skin anti-aging agent such as an anti-wrinkle agent.

As the skin care active ingredient, mention may be made of Vitamin B3 and derivatives, ascorbic acid and derivatives thereof, resorcinol derivatives, C-glycoside derivatives, salicylic acid and derivatives thereof, α-hydroxy acids, niacinamide and mixtures thereof.

The skin care active ingredient(s) may be present in the composition in a content of 0.3% by weight or more, preferably 1% by weight or more, and more preferably 3% by weight or more, and it may be present in the composition in a content of 15% by weight or less, preferably 10% by weight or less, and more preferably 7% by weight or less, relative to the total weight of the composition.

The amount of the skin care active ingredient(s) in the composition according to the present invention may be from 0.3% to 15% by weight, preferably from 1% to 10% by weight, and more preferably from 3% to 7% by weight, relative to the total weight of the composition.

### Additives

The composition according to the present invention may also comprise any other optional additive(s) usually used in the field of cosmetics, chosen, for example, from cationic, nonionic, anionic, or amphoteric polymers, cationic, nonionic or amphoteric surfactants, hydrophobic organic solvents, gums, dyes, resins, thickeners, dispersants, antioxidants, preserving agents such as phenoxyethanol, fragrances, neutralizers, pH adjusting agents, antiseptics, other cosmetic active agents, vitamins such as tocopherol, moisturizers, chelating agents, emollients or collagen-protecting agents, and mixtures thereof.

The composition according to the present invention can be prepared by mixing the above-described essential and optional ingredients in a conventional manner. In the case that at least one of the above ingredients is solid at room temperature, the ingredient can be heated until it is dissolved. It is possible to further comprise mixing any of the optional ingredients and heating the composition until the ingredient is dissolved.

### [Cosmetic Process]

The composition according to the present invention is intended to be used as a cosmetic composition. Therefore, the cosmetic composition according to the present invention may be intended for application onto keratin substances, for example the skin, scalp, hair, mucosa such as lips, and nails, in particular the skin, for instance that of the face.

The composition according to the present invention may be used as a skin cosmetic composition, preferably a skin makeup composition or a skin care composition, more preferably a skin makeup composition, and in particular a foundation.

Thus, the present invention also relates to a non-therapeutic cosmetic method or process, preferably for caring for and/or conditioning keratinous substance, comprising: applying onto the keratinous substance, such as the skin, scalp, and lips, in particular facial skin a composition having at least one aqueous phase and at least one oily phase, comprising:
(a) at least one spherical hydrophobic silica aerogel;
(b) at least one lipophilic thickener;
(c) at least one hydrophobic surface-treated pigment;
(d) at least one nonionic surfactant selected from esters of polyols with fatty acids;
(e) at least one polyol;
(f) at least one monoalcohol; and
(g) at least one anionic polymer,
   wherein the aqueous phase is present in an amount of 30% by weight or more relative to the total weight of the composition.

The same explanations given for the composition, the (a) at least one spherical hydrophobic silica aerogel; (b) at least one lipophilic thickener; the (c) at least one hydrophobic surface-treated pigment; the (d) at least one nonionic surfactant selected from esters of polyols with fatty acids; the (e) at least one polyol; the (f) at least one monoalcohol; and the (g) at least one anionic polymer can be applied to those for the process according to the present invention. The composition used in the process according to the present invention may include any of the optional ingredients explained above for the composition according to the present invention.

### EXAMPLES

The present invention will be described in a more detailed manner by way of examples. However, these examples should not be construed as limiting the scope of the present invention.

### Preparation Method of Inventive and Comparative W/O Foundation Compositions

The ingredients listed as A1 in Tables 1 and 2 were completely mixed at 45° C. Disteardimonium hectorite listed as A2 was then added and they were mixed with a Moritz homogenizer at 3,500 rpm for 5 minutes at 45° C. The ingredients as listed C were added and mixed at 3,500 rpm for 10 minutes at 45° C. A mixture of the ingredients was cooled down to 25° C. The ingredients listed as D was added and mixed at 3,000 rpm for 2 minutes at room temperature, and then the ingredients listed as E was added and dispersed at 2,000 rpm for 5 minutes at room temperature to obtain the emulsion compositions.

The spherical silica silylate aerogel was obtained from Tokuyama and had 10 μm of the average primary particle size, 0.88 of an average circularity, 592 m²/g of BET specific surface area, 4.0 ml/g of a pore volume determined by BJH method, 6.8 mL/g of an oil absorption capacity measured with JIS-K6217-4, and 20 nm of a peak pore radius determined by BJH method.

### Evaluation

### [Texture Assessments]

10 professional panelists evaluated the sensory aspects of “water-splashing sensation”, “moist sensation”, and “moisture lastingness” in accordance with the following criteria. “Water-splashing sensation” was assessed during application of the composition on the face. “Moist sensation” was assessed 5 minutes after the composition was applied on the face. “Moisture lastingness” was assessed 6 hours after the composition was applied on the face.

### (Moist Sensation)

Excellent: All of 5 professional panels felt moist sensation.

Very good: 4 panels of 5 professional panels felt moist sensation.

Good: 3 panels of 5 professional panels felt moist sensation.

Fair: 2 panels of 5 professional panels felt moist sensation.

Bad: 0-1 panel of 5 professional panels felt moist sensation.

### (Water-Splashing Sensation)

Excellent: All of 5 professional panels felt water-splashing sensation.

Very good: 4 panels of 5 professional panels felt water-splashing g sensation.

Good: 3 panels of 5 professional panels felt water-splashing sensation.

Fair: 2 panels of 5 professional panels felt water-splashing sensation.

Bad: 0-1 panel of 5 professional panels felt water-splashing sensation.

### (Moisture Lastingness)

Excellent: All of 5 professional panels felt moisture at 6 hours after application.

Very good: 4 panels of 5 professional panels felt moisture at 6 hours after application.

Good: 3 panels of 5 professional panels felt moisture at 6 hours after application.

Fair: 2 panels of 5 professional panels felt moisture at 6 hours after application.

Bad: 0-1 panel of 5 professional panels felt moisture at 6 hours after application.

### [Stability]

### (Centrifuge Test)

A centrifuge test was used to determine how stable the formulation was in the above compositions. Separation after centrifuging indicates instability, and is thus not generally desired. 7 mL of each compositions was transferred into a 15 mL plastic centrifuge tube. The samples were centrifuged for 60 minutes at 3000 rpm using KUBOTA Compact Tabletop Centrifuge 2420. The samples were then evaluated for oil separation in accordance with the following criteria. “N/A” in the tables indicates that the test result was not available.

Very good: No separation was observed for two months with viscosity reduction.

Good: No separation was observed for one month with no viscosity reduction.

Fair: No separation was observed for one month with viscosity reduction.

Bad: Separation was observed after one day.

### (Vibration Test)

A vibration test was used to determine the stability of the pigments in the above formulations. Streaks of pigments on the inside of the glass jar indicate instability of the pigments in the formulation and is thus not generally desired. Each composition was filled in 50 mL glass bottle and put in 45 Celsius degree incubator for one night. The samples were directly put under vibration for 60 minutes at 180 rpm using AS ONE Neo-Shaker NS-LR. The samples were then evaluated for aggregation of pigments by observing appearance of the samples. “N/A” in the tables indicates that the test result was not available.

Excellent: No streaks or dots of pigments were observed.

Very good: Very small dots of pigments were observed.

Good: Small dots of pigments were observed.

Fair: Streaks of pigments were observed.

Bad: Blotches of pigments were observed.

The results are shown in Tables 1 and 2.

TABLE 1

| Ingredient | | Ex.1 | Ex. 2 | Ex. 3 | Ex.4 | Ex.5 | Ex.6 |
|---|---|---|---|---|---|---|---|
| A1 | PEG-10 Dimethicone | 2 | 2 | 2 | 2 | 1.5 | 2 |
| | Sorbitan isostearate | 2.5 | 2 | 1.5 | 1 | 1.5 | 1.5 |
| | Bis-PEG/PPG-14/14 Dimethicone | 1 | 1 | 1 | 1 | 1 | 1 |
| | (and) Dimethicone | | | | | | |
| | Dimethicone | 8 | 8 | 8 | 8 | 8 | 8 |
| | Undecane (and) Tridecane | 7 | 7 | 7 | 7 | 7 | 7 |
| | Phenyl trimethicone | 2 | 2 | 2 | 2 | 2 | 2 |
| | Diisopropyl sebacate | 2.47 | 2.47 | 2.47 | 2.47 | 2.47 | 2.47 |
| | Dicaprylyl carbonate | 6.18 | 6.18 | 5.68 | 6.18 | 6.18 | 5.68 |
| | Macademia nut oil | 2 | 2 | 2 | 2 | 2 | 2 |
| | Tocopherol | 0.11 | 0.11 | 0.11 | 0.11 | 0.11 | 0.11 |
| | Trimethylsiloxysilicate | 6.67 | 6.67 | 6.67 | 6.67 | 6.67 | 6.67 |
| A2 | Disteardimonium hectorite | 1.2 | 1.2 | 1.2 | 1.2 | 1.2 | 1.2 |
| B | Perlite | 0.1 | 0.1 | 0.1 | 0.1 | 0.1 | 0.1 |
| | Silica silylate | 0.5 | 0.5 | 0.5 | 0.5 | 0.5 | 0.5 |
| | Silica (and) Titanium dioxide | 0.1 | 0.1 | 0.1 | 0.1 | 0.1 | 0.1 |
| C | Glycerin | 5 | 5 | 5 | 5 | 5 | 5 |
| | Butylene glycol | 8 | 8 | 8 | 8 | 8 | 8 |
| | Phenoxyethanol | 0.5 | 0.5 | 0.5 | 0.5 | 0.5 | 0.5 |
| | Sodium hyaluronate (Mw: 35,000) | 1 | 1 | 1 | 1 | 1 | 1 |
| | Water | qs 100 | qs 100 | qs 100 | qs 100 | qs 100 | qs 100 |
| | Niacinamide | 5 | 5 | 5 | 5 | 5 | 5 |
| | Magnesium sulfate | 0.3 | 0.3 | 0.3 | 0.3 | 0.3 | 0.3 |
| D | Ethanol | 6.5 | 6.5 | 6.5 | 6.5 | 6.5 | 6.5 |
| E | Titanium dioxide (and) Alumina | 12.62 | 12.62 | 12.62 | 12.62 | 12.62 | 7.95 |
| | (and) Dimethicone (and) Isopropyl | | | | | | |
| | titanium triisostearate | | | | | | |
| | Iron oxides (and) Isopropyl | 1.94 | 1.94 | 1.94 | 1.94 | 1.94 | 6.61 |
| | titanium triisostearate | | | | | | |
| Water Phase Content (wt %) | | 37.11 | 37.61 | 38.61 | 38.61 | 38.61 | 38.61 |
| Texture | Moist sensation | very | very | very | very | very | very |
| Assessments | | good | good | good | good | good | good |
| | Water-splashing | very | very | very | very | very | very |
| | sensation | good | good | good | good | good | good |
| | Moisture | very | very | very | very | very | very |
| | lastingness | good | good | good | good | good | good |
| Stability | Centrifuge | very | very | very | very | very | very |
| | | good | good | good | good | good | good |
| | Vibration test | excell- | excell- | very | good | very | very |
| | | ent | ent | good | | good | good |

TABLE 2

| | | | | | | |
|---|---|---|---|---|---|---|
| Comp. | Comp. | Comp. | Comp. | Comp. | Comp. | Comp. |

| Ingredient | | Ex.1 | Ex.2 | Ex.3 | Ex.4 | Ex.5 | Ex.6 | Ex.7 |
|---|---|---|---|---|---|---|---|---|
| A1 | PEG-10 Dimethicone | 2 | 2 | 2 | 0.4 | — | 0.4 | 0.4 |
| | Sorbitan isostearate | — | 1 | 1 | 1.6 | 1.6 | 1.6 | 1.6 |
| | Bis-PEG/PPG-14/14 Dimethicone | 1 | 1 | 1 | — | — | — | — |
| | (and) Dimethicone | | | | | | | |
| | PEG-30 Dipolyhydroxystearate | — | — | — | 2.2 | 2.2 | 2.2 | 2.2 |
| | Dimethicone | 8 | 8 | 8 | 9.2 | 10.1 | 9.7 | 15.7 |
| | Undecane (and) Tridecane | 7 | 7 | 7 | 6.6 | 6.6 | 6.6 | 6.6 |
| | Phenyl trimethicone | 2 | 2 | 2 | 2 | 1 | 2 | 2 |
| | Diisopropyl sebacate | 2.47 | 2.47 | 2.47 | 1.6 | 1.6 | 1.6 | 1.6 |
| | Dicaprylyl carbonate | 6.18 | 6.18 | 6.18 | — | — | — | — |
| | Macademia nut oil | 2 | 2 | 2 | — | — | — | — |
| | Isododecane | — | — | — | — | 1.1 | — | — |
| | Ethylhexyl salicylate | — | — | — | 5 | 3.3 | 5 | 5 |
| | Octocrylene | — | — | — | — | 3.3 | — | — |
| | Homosalate | — | — | — | 5 | 4.4 | 5 | 5 |
| | Tocopherol | 0.11 | 0.11 | 0.11 | 0.1 | 0.1 | 0.1 | 0.1 |
| | Trimethylsiloxysilicate | 6.67 | 6.67 | 6.67 | 6.7 | 7.3 | 6.7 | 6.7 |
| A2 | Disteardimonium hectorite | 1.2 | 1.2 | 1.2 | 1.2 | 1.2 | 1.2 | 1.2 |
| B | Perlite | 0.1 | 0.1 | 0.1 | 0.1 | 1.1 | — | 0.1 |
| | Silica silylate | 0.5 | 0.5 | 0.5 | 0.5 | 0.9 | — | 0.5 |
| | Silica (and) Titanium dioxide | 0.1 | 0.1 | 0.1 | 0.1 | 3.3 | — | 0.1 |
| | Silica | — | — | — | — | 0.1 | — | — |
| C | Glycerin | 5 | 5 | 5 | 5 | 3.3 | 5 | 5 |
| | Butylene glycol | 8 | 8 | 8 | 8 | 2.2 | 8 | 8 |
| | Phenoxyethanol | 0.5 | 0.5 | 0.5 | 0.5 | 0.5 | 0.5 | 0.5 |
| | Sodium hyaluronate (Mw: 35,000) | 1 | 1 | 1 | — | — | 1 | 1 |
| | Water | qs 100 | qs 100 | qs 100 | qs 100 | qs 100 | qs 100 | qs 100 |
| | Niacinamide | 5 | 5 | 5 | 5 | — | 5 | 5 |
| | Magnesium sulfate | 0.3 | 0.3 | 0.3 | 0.3 | 0.8 | 0.3 | 0.3 |
| D | Ethanol | 6.5 | 6.5 | — | 6.8 | 7.8 | 6.8 | 6.8 |
| E | Titanium dioxide (and) Alumina | 12.62 | — | 12.62 | 12.6 | 12.7 | 12.6 | 12.6 |
| | (and) Dimethicone (and) Isopropyl | | | | | | | |
| | titanium triisostearate | | | | | | | |
| | Iron oxides (and) Isopropyl titanium | 1.94 | — | 1.94 | 2 | 2 | 2 | 2 |
| | triisostearate | | | | | | | |
| | Titanium dioxide | — | 12.62 | — | — | — | — | — |
| | Iron oxide | — | 1.94 | — | — | — | — | — |

| Water Phase Content (wt %) | | 39.61 | 38.61 | 45.11 | 36.3 | 29.6 | 36.5 | 29.8 |
|---|---|---|---|---|---|---|---|---|
| Texture | Moist sensation | fair | good | fair | very | fair | very | very |
| Assessments | | | | | good | | good | good |
| | Water-splashing | fair | good | fair | fair | fair | very | fair |
| | sensation | | | | | | good | |
| | Moisture | very | very | very | very | fair | bad | very |
| | lastingness | good | good | good | good | | | good |
| Stability | Centrifuge | good | bad | fair | N/A | N/A | N/A | N/A |
| | Vibration test | fair | bad | bad | N/A | N/A | N/A | N/A |

As shown in Tables 1 and 2 above, the compositions according to Examples 1 to 6 which include the combinations of ingredients (a) to (g) and an aqueous phase in an amount of 30% by weight or more relative to the total weight of the composition showed good sensory properties with respect to “water-splashing sensation”, “moist sensation”, and “moisture lastingness”. In addition, these composition exhibited a good stability with a pigment dispersibility.

On the other hand, the composition according to Comparative Example 1, which did not include the ionic surfactant of the present invention, showed inadequate sensory properties and pigment stability. The composition according to Comparative Example 2, which did not include the hydrophobic surface-treated pigment, showed poor phase and pigment stability. The composition according to Comparative Example 3, which did not include the monoalcohol, showed inadequate sensory properties and phase stability, and poor pigment stability. The composition according to Comparative Example 4, which did not include the anionic polymer, did not provide an improved water-splashing sensation. The composition according to Comparative Example 5, which did not include the anionic polymer and included less than 30% by weigh of the aqueous phase, showed inadequate sensory properties. The composition according to Comparative Example 6, which did not include the spherical hydrophobic silica aerogel of the present invention, showed poor moisture lastingness. The composition according to Comparative Example 7, which included less than 30% by weight of the aqueous phase, did not provide an adequate water-splashing sensation.

Therefore, it can be concluded that the composition according to the present invention has a great benefit since it can provide excellent cosmetic properties, such as providing a fresh and moist feeling, as well as, good moisture lastingness while it exhibits a good stability of a formulation. Therefore the compositions according to the present invention are very useful as cosmetic compositions for keratinous substances, such as skin, and in particular as foundations.

1. A cosmetic emulsion composition having at least one aqueous phase and at least one oily phase, comprising:
(a) at least one spherical hydrophobic silica aerogel;
(b) at least one lipophilic thickener;
(c) at least one hydrophobic surface-treated pigment;
(d) at least one nonionic surfactant other than nonionic silicone surfactants;
(e) at least one polyol;
(f) at least one monoalcohol; and
(g) at least one anionic polymer,
wherein the aqueous phase is present in an amount of 30% by weight or more relative to the total weight of the composition.

2. The cosmetic composition according to claim 1, wherein the spherical hydrophobic silica aerogel is a spherical hydrophobic aerogel of silica silylate.

3. The cosmetic composition according to claim 1, wherein the spherical hydrophobic silica aerogel has an average circularity determined by an image analysis method of 0.8 or more.

4. The cosmetic composition according to claim 1, wherein the lipophilic thickener is selected from mineral lipophilic thickeners.

5. The cosmetic composition according to claim 1, wherein the hydrophobic surface-treated pigment is coated with isopropyl titanium triisostearate.

6. The cosmetic composition according to claim 1, wherein the polyol comprises at least one diol and at least one polyol having three or more —OH functions in combination.

7. The cosmetic composition according to claim 1, wherein the polyol comprises at least 3% by weight of at least one diol and at least 3% by weight of at least one polyol having three or more —OH functions in combination, relative to the total weight of the composition.

8. The cosmetic composition according to claim 1, wherein a number average molecular weight of the anionic polymer ranges from 1,000 to 1,000,000.

9. The cosmetic composition according to claim 1, wherein the anionic polymer is selected from the group consisting of polysaccharides, hyaluronic acid and derivatives thereof, and cellulose polymers, anionic (co)polyaminoacids, (co)poly(meth)acrylic acids, (co)polyamic acids, (co)polystyrene sulfonate, (co)poly(vinyl sulfate), dextran sulfate, chondroitin sulfate, (co)polymaleic acids, (co)polyfumaric acids, maleic acid (co)polymers, and salts thereof.

10. The cosmetic composition according to claim 1, wherein the anionic polymer is selected from hyaluronic acid and derivatives thereof, and a salt thereof.

11. The cosmetic composition according to claim 1, wherein the nonionic surfactant is selected from esters of polyols with fatty acids.

12. The cosmetic composition according to claim 1, further comprising at least one film-forming polymer.

13. The cosmetic composition according to claim 1, further comprising at least one thickening aid selected from polyoxyalkylenated silicone and alkyl or alkylene carbonate.

14. The cosmetic composition according to claim 1, wherein the cosmetic composition is a skin makeup or a skin care composition.

15. A cosmetic process for a keratin substance, comprising applying the cosmetic composition according to claim 1 to the keratin substance.
